(19) United States
(12) Patent Application Publication
(10) Pub. No.: US 2024/0041786 A1
(43) Pub. Date: Feb. 8, 2024

(54) AMINO ALCOHOL IONIZABLE LIPIDS

(71) Applicant: NEW JERSEY INSTITUTE OF TECHNOLOGY, Newark, NJ (US)

(72) Inventors: Xiaoyang Xu, Livingston, NJ (US); Zhongyu Li, Harrison, NJ (US)

(73) Assignee: NEW JERSEY INSTITUTE OF TECHNOLOGY, Newark, NJ (US)

(21) Appl. No.: 18/209,195

(22) Filed: Jun. 13, 2023

Related U.S. Application Data
(60) Provisional application No. 63/390,751, filed on Jul. 20, 2022.

(51) Int. Cl. A61K 9/51; A61K 45/06; A61K 48/00

(52) U.S. Cl. CPC A61K 9/5123; A61K 45/06; A61K 9/5192; A61K 48/0091; B82Y 5/00

(57) ABSTRACT

Ionizable cationic lipid compounds have an amine moiety from amino alcohols and a lipid moiety from a lipid synthesized via esterification. The ionizable cationic lipid compounds which comprise an amino alcohol mediated ionizable cationic lipid compound are useful for in vivo or in vitro delivery of one or more nucleic acid agents including DNA, siRNA, a microRNA, an mRNA, a RNAi, and a plasmid.

### CROSS-REFERENCE TO RELATED APPLICATION

The present application claims the benefit of priority to U.S. Provisional Appl. No. 63/390,751, filed Jul. 20, 2022, which is hereby incorporated by reference in its entirety.

### STATEMENT REGARDING FEDERALLY SPONSORED RESEARCH

This invention was made with government support under Agreement No. 19AIREA34380849 awarded by the American Heart Association. This invention was made with government support under Grant No. 2001606 awarded support by the National Science Foundation. The government has certain rights in the invention.

### FIELD

The present disclosure relates to gene therapy. In particular, the present disclosure relates to the synthesis, development, and use of amino alcohol mediated ionizable cationic lipids and their nanoparticle-based formulations in nucleic acid-based gene delivery and therapy. Ionizable cationic lipids can be formulated into lipid nanoparticles (LNPs) which are capable to bind to and encapsulate negatively charged nucleic acid-based genes.

### BACKGROUND

Severe acute respiratory syndrome coronavirus 2 (SARS-CoV-2) is the catalyst of the catastrophic global COVID-19 pandemic which has infected over 270 million people, resulting in over 5.3 million deaths worldwide as reported by the World Health Organization as of December 2021. The two mRNA-based COVID-19 vaccines from Pfizer-BioNTech (BNT162b2) [1, 2] and Moderna (mRNA-1273) [3, 4] are manufactured using lipid nanoparticles (LNPs), and their success truly solidified the significant impact of mRNA-LNPs therapy as a groundbreaking technology in human public health [5]. Therefore, this opens up enormous avenues for mRNA gene therapies to treat most diseases by expressing therapeutic proteins [6]. LNPs have emerged as the most clinically advanced non-viral gene delivery system which can deliver siRNA and mRNA safely and effectively to overcome the major barrier preventing the development of genetic therapy [7-10]. In general, the FDA-approved LNP formulations contain ionizable cationic lipids, phospholipids, cholesterol, and PEGylated lipids, each with specialized functions indispensable to RNA delivery [5-8, 11]. It is important to note that the ionizable cationic lipid is the most essential component, specifically because it determines the mRNA transfection efficacy and by extension the therapeutic outcomes [6-8]. The ionizable cationic lipids mediated mRNA-LNP delivery platforms have shown great potential in the field of nanoparticle-mediated vaccines [1, 3], gene editing [12-14] and cancer therapy [15,16] nowadays. Consequently, to fully realize the effectiveness of mRNA therapeutics, developing high-performing ionizable cationic lipids for mRNA delivery is of great urgency and importance for the next generation therapeutics [5].

The previous FDA-approved ionizable cationic lipids such as DLin-MC3-DMA (MC3) [9], ALC-0315 [17], SM-102 are synthesized through multiple-step reactions, with drawback such as a low chemical yield after multiple post-reaction processes [9, 18]. Moreover, most of the published lipid-like materials were synthesized via Michael Addition between the amino and acrylate groups or ring-opening of epoxides with amines, which made the resulting lipid-like materials non-biodegradable and with potential cytotoxicity [12, 19-22]. To this end, an aim of this disclosure was to develop a new and high-efficient synthesis strategy of biodegradable ionizable cationic lipids for mRNA-LNPs delivery platform. Enzyme-assisted chemical reactions have been widely employed in various laboratory and industrial processes since the enzymes are non-toxic, recyclable, and eco-friendly biocatalysts; in particular, Candida antarctica Lipase B-immobilized on the acrylic resin (CALB) is the most frequently used, high-efficiency biocatalyst in organic syntheses such as esterification and transesterification [23-26].

### SUMMARY

Herein, a new library of ionizable cationic lipids has been designed and synthesized through a one-step Candida antarctica Lipase B-immobilized on the acrylic resin (CALB) enzyme-assisted high-efficiency esterification between amino alcohols and commercial-available lipid acids, creating an 18*8 library of lipid-like materials. Through high-throughput screening, a desirable ionizable lipid was determined to be AA3-DLin which was designed with (1) 1,4-Bis(2-hydroxyethyl) piperazine as the hydrophilic headgroup, (2) two linoleic hydrocarbon chains promoting self-assembly and (3) two degradable ester bonds as linkers connecting the headgroups with hydrocarbon chains, which lowers potential systemic cytotoxicity [9, 10]. Advantageously, the one-step CALB-mediated esterification led to a high-throughput synthesis of lipid-like materials library with at least 85% chemical yield and high product purity, especially, the AA3-DLin showed a ˜96% purity in a one-step esterification with undetectable by-products. To further investigate the AA3-DLin LNP formulation, orthogonal design of experiment (DoE) methodology [22,27] was employed to fine-tune the molar ratios of AA3-DLin, DOPE, cholesterol, and DMG-PEG. The A12 formulation of AA3-DLin LNP has been demonstrated a 6-fold higher transfection efficacy over MC3 [6, 9] and a 3-fold over lipofectamine 3000 in vitro, as well as excellent transfection efficacy in vivo. Notably, the AA3-DLin LNPs presented outstanding thermostability which can maintain mRNA delivery efficacy at 4° C. for at least one week and excellent capability of long-term storage which allowed the mRNA-LNPs formulation to be stored for at least 12 months at −20° C. without a reduction in transfection efficacy.

In accordance with embodiments of the present disclosure, the new chemical structures of ionizable cationic lipids are disclosed. The ionizable cationic lipids are synthesized by amino alcohols and lipid acids (also known as fatty acids). Specifically disclosed are amino alcohol mediated ionizable cationic lipid compounds, which in one or more embodiments are the reaction products of an amino alcohol and a lipid acid, and 18 different amino alcohols and 8 different lipid acids are exemplified. Therefore, 18*8=144 different chemical structures of amino alcohol mediated ionizable cationic lipid compounds are being disclosed, suitable for gene delivery. FIG. 5 schematically discloses the chemical structures of the reaction ingredients: where the R represents various lipid acid aliphatic chains of an acid (COOH) (representing lipid acid carbon contents of C8 to C18 shown in FIG. 3) suitable for synthesis with various amino alcohols (structures with assigned numbers 1 to 18 of FIG. 5). In particular, the amino alcohol mediated ionizable cationic lipid compound named “AA3-DLin” which is synthesized via esterification of 1,4-Bis(2-hydroxyethyl) piperazine and linoleic acid is disclosed as the top-performing amino alcohol mediated ionizable cationic lipid compound.

In an aspect, a lipid nanoparticle (LNP) contains four parts: 1) ionizable cationic lipid, 2) phospholipid, 3) cholesterol and 4) PEGylated lipid self-assemble to form nano particles to delivery encapsulated gene payloads such as siRNA, miRNA, mRNA and DNA plasmid. In one embodiment, the AA3-DLin was formulated with 1,2-dioleoyl-sn-glycero-3-phosphoethanolamine (DOPE), cholesterol, and 1,2-dimyristoyl-rac-glycero-3-methoxypolyethylene glycol (DMG-PEG) to form AA3-DLin LNP structures. In one or more embodiments, the AA3-DLin LNP structures comprise unique molar ratios of amino alcohol mediated ionizable cationic lipid compound to DOPE to cholesterol to PEGylated lipid (e.g. DMG-PEG). In one or more embodiments of the AA3-DLin LNP structure, a molar percentage of AA3-DLin is in a range of from 20-60%, a molar percentage of DOPE is in a range of from 20-50%, a molar percentage of cholesterol is in a range of from 20-50%, and a molar percentage of PEGylated lipid (e.g. DMG-PEG) is in a range of from 0.1-5%. In one or more embodiments, a total of the molar percentages of the AA3-DLin, DOPE, cholesterol, and DMG-PEF is 100%. In a particular embodiment, a formulation of a LNP structure comprises a molar ratio of 40:40:25:0.5 of AA3-DLin:DOPE:cholesterol:DMG-PEG, which is the effective for in vivo and in vitro gene delivery.

In one embodiment, the AA3-DLin LNP structures are fabricated using a microfluidic chip device where the water phase contained a gene payload of interest and an organic phase contained an ethanol mixture of AA3-DLin, DOPE, cholesterol and DMG-PEG with a molar ratio of 40:40:25:0.5. In particular, by adjusting flow rates (of syringe pumps), particle size of the LNP structures can be controlled in a range of 50-300 nm.

In previous publications, FDA-approved ionizable cationic lipids such as DLin-MC3-DMA (MC3) [9], ALC-0315 [17], SM-102 are synthesized through multiple-step reactions, with drawback such as a low chemical yield after multiple post-reaction processes [9, 18]. Moreover, most of the published lipid-like materials were synthesized via Michael Addition between the amino and acrylate groups or ring-opening of epoxides with amines, which made the resulting lipid-like materials non-biodegradable and with potential cytotoxicity [12, 19-22]. To this end, an objective herein was to develop a new and high-efficient synthesis strategy of biodegradable ionizable cationic lipids for mRNA-LNPs delivery platform. Enzyme-assisted chemical reactions have been widely employed in various laboratory and industrial processes since the enzymes are non-toxic, recyclable, and eco-friendly biocatalysts; in particular, Candida antarctica Lipase B-immobilized on the acrylic resin (CALB) is the most frequently used, high-efficiency biocatalyst in organic syntheses such as esterification and transesterification [23-26].

In one embodiment, a synthesis method using an enzyme-immobilized on acrylic resin esterification for synthesizing the ionizable cationic lipids, which is further used in LNP formulations, is disclosed. In one or more embodiments, the enzyme comprises but is not limited to Candida antarctica Lipase B (CALB).

General aspects herein include lipid nanoparticles (LNPs) comprising a new class of ionizable cationic lipids, which comprise: an ionizable cationic lipid compound comprising a reaction product of: an amino alcohol and one or more lipid acids having from 4 to 26 carbons (C4-C26); and one or more other lipid components selected from the group consisting of: a helper neutral lipid, a PEG-modified lipid, and/or cholesterol. Exemplified structures are in accordance with the reaction ingredients of FIG. 5, discussed herein. Other aspects include therapeutic lipid nanoparticles comprising: a lipid phase and a nucleic acid agent, the lipid phase comprising any LNP disclosed herein. Further aspects include lipid nanoparticle delivery systems comprising a plurality of any of the therapeutic lipid nanoparticles disclosed herein.

Any combination and/or permutation of the following embodiments is envisioned. Other objects and features will become apparent from the following detailed description considered in conjunction with the accompanying drawings. It is to be understood, however, that the drawings are designed as an illustration only and not as a definition of the limits of the present disclosure.

### BRIEF DESCRIPTION OF THE DRAWINGS

To assist those of skill in the art in making and using the disclosed amino alcohol mediated ionizable cationic lipids and associated systems and methods, reference is made to the accompanying figures, wherein:

FIG. 1 shows a chemical synthesis route of AA3-DLin through CALB enzyme-assisted esterification according to one or more embodiments;

FIG. 2 illustrates schematic views of an ionizable cationic lipid and an assembled ionizable cationic lipid nanoparticle (LNP), according to one or more embodiments;

FIG. 3 shows chemical structures of amino alcohols and lipid acids used in the synthesis of lipid-like materials according to one or more embodiments;

FIG. 4 shows luciferase expression heat map of mLuc-loaded LNPs. The LNPs were prepared with different lipid-like materials where the x-axis represents different amino alcohols and the y-axis represents different lipid acids used for synthesis;

FIG. 5 schematically discloses reaction ingredients suitable to prepare the 18*8=144 different chemical structures of amino alcohol mediated lipid compounds, where the R represents 8 different lipid acid aliphatic chains of the acids shown in FIG. 3 for grafting on 18 different amino alcohols according to one or more embodiments;

FIG. 6 is a schematic view illustrating 4 components of AA3-DLin LNPs for Design of Experiment (DoE) calculation. The organic phase contains AA3-DLin, DOPE, cholesterol, and DMG-PEG at a molar ratio of 40:40:25:0.5, according to one or more embodiments. The water phase contains an mRNA of interest (in NaOAc buffer) according to one or more embodiments. Two L₁₆ (4⁴) orthogonal tables were employed to guide preparation of LNP formulations;

FIG. 7 is a schematic view of a therapeutic lipid nanoparticle comprising a lipid nanoparticle (LNP) encapsulating a nucleic acid agent according to one or more embodiments. The LNP comprises an outer layer or shell, which is in this embodiment is composed of four components (AA3-DLin, DOPE, cholesterol, DMG-PEG 2000) that are randomly assembled, which encapsulate an mRNA via electrostatic interactions, according to one or more embodiments;

FIGS. 8 and 9 show the transfection efficacy and cell viability of different AA3-DLin LNP formulations in Library A and Library B (as well as lipofectamine 3000 and MC3), which were evaluated on Hek 293 cells;

FIG. 10 shows a schematic illustration of LNP fabrication through microfluidic mixing;

FIG. 11 shows the TEM image of AA3-DLin LNPs stained by uranyl acetate solution (scale bar: 100 nm);

FIG. 12 shows the characterization summary of LNPs prepared under high pump rates;

FIG. 13 shows pKa value of AA3-DLin LNPs measured by TNS fluorescence titration. pKa was defined as the pH at half-maximal fluorescence intensity. All experiments were repeated in triplicate to generate results and data are shown as mean±SD;

FIG. 14A shows fluorescence imaging and FIGS. 14B-14D show flow cytometry results of Hek 293 cells transfected by mCherry-LNPs at different time points;

FIG. 15 shows a summary of mCherry positive cell population transfected by different formulations;

FIG. 16 shows mean fluorescence intensity (MFI) of mCherry positive cells according to flow cytometry data;

FIG. 17 shows the transfection evaluation of 6-month stored mCherry-LNPs in vitro, the data were summarized by flow cytometry and normalized to fresh LNPs;

FIG. 18 shows summary of luciferase expression generated by different long-term stored mLuc-LNP formulations in vivo, the luciferase expression was recorded after 6 hours post-administration;

FIG. 19 shows the comparison of particle size and distribution between fresh LNPs and 6-month frozen stored LNPs;

FIGS. 20 and 21 show in vivo bioluminescence images (total flux) of BALB/c mice after administration of fresh mLuc-LNPs and 6-month frozen mLuc-LNPs (n=3) at specific time points;

FIG. 22 shows in vivo transfection efficacy comparison between fresh mLuc-LNPs, 6-month frozen mLuc-LNPs, and 6-month lyophilized mLuc-LNPs. The luciferase expression was recorded for 2 weeks;

FIG. 23 shows the mLuc-LNPs were stored at 4° C. or 25° C. (room temperature) for 1, 3, 5, 7, and 9 days before injecting to mice. The luciferase expression was recorded after 6 hours post-administration and normalized to fresh mLuc-LNPs. All experiments were repeated in triplicate and the data were represented as means±SD;

FIG. 24 is an electrospray ionization (ESI) mass spectrometry graph of relative abundance versus m/z for synthesis of AA3-DLin with molecular weight (MW) of 699 and up to 96% purity;

FIG. 25 is a nuclear magnetic resonance (NMR) spectroscopy graph showing the chemical structure of AA3-DLin; and

FIG. 26 is a Fourier-transform infrared spectroscopy (FTIR) graph depicting % transmittance versus wavenumbers (cm⁻¹) for the chemical structure of AA3-DLin.

### DETAILED DESCRIPTION

Exemplary embodiments are directed to amino alcohol mediated ionizable cationic lipid nanoparticles (AA-LNPs) platform. It should be understood that embodiments can generally be applied to other nucleic acid agents situated within or encapsulated within the AA-LNPs.

### Results and Discussions

Design, Synthesis, and Screening of Ionizable Cationic Lipid Library

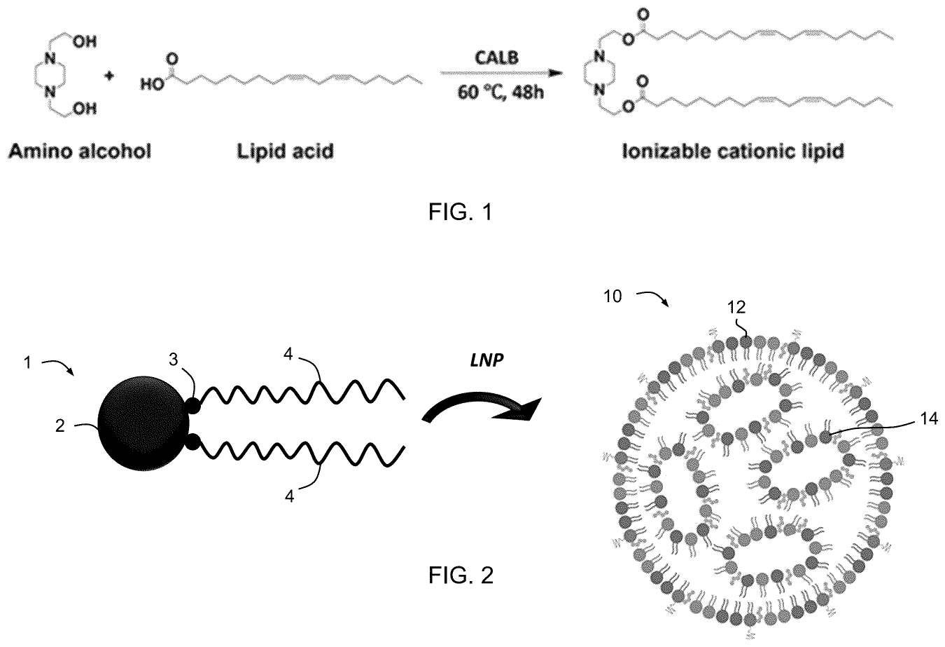

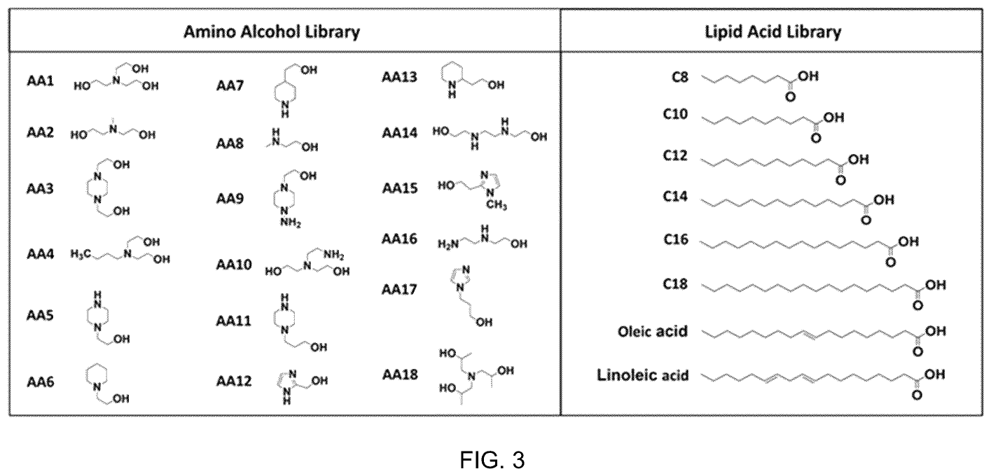

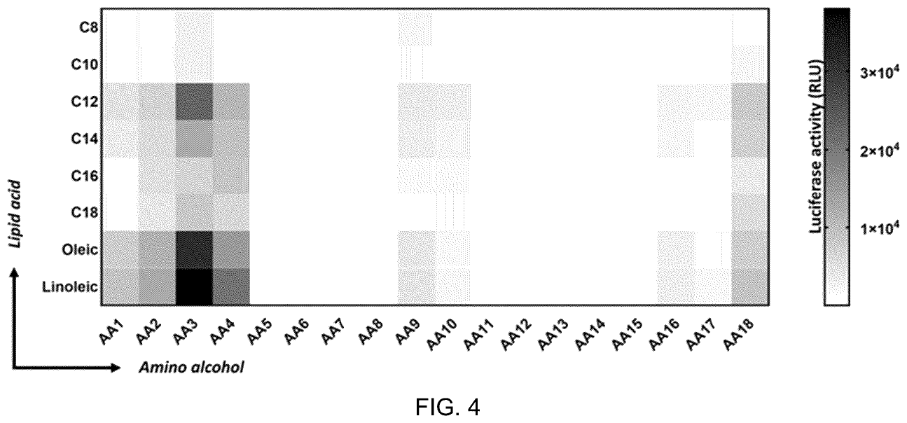

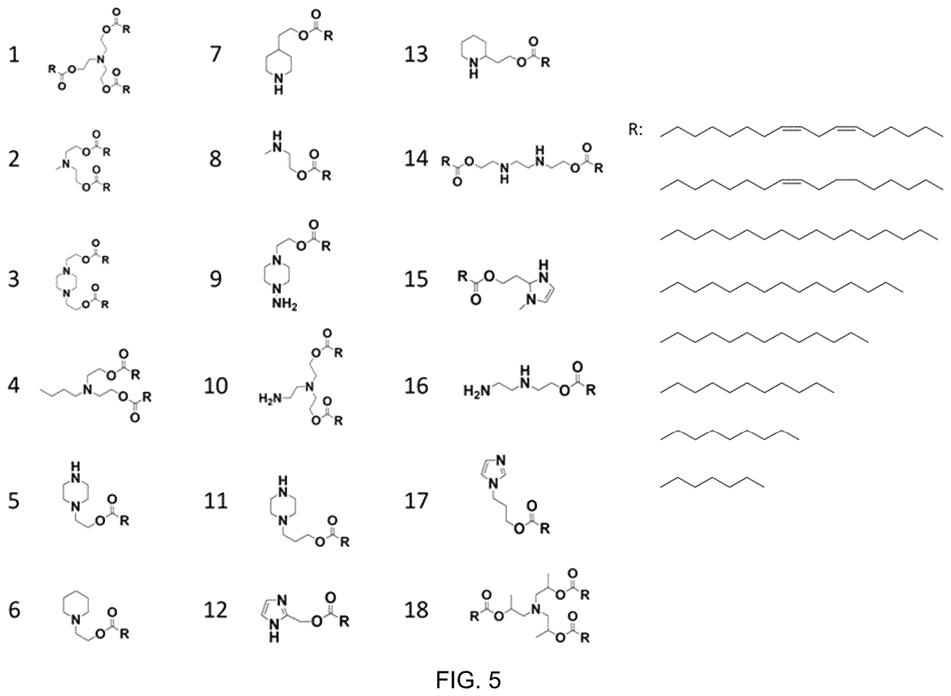

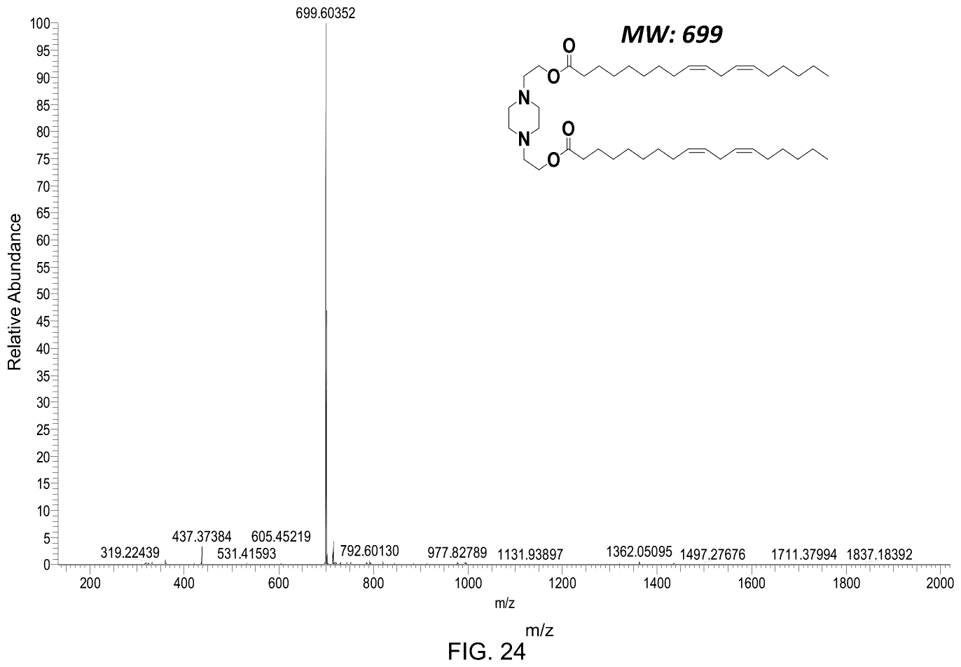

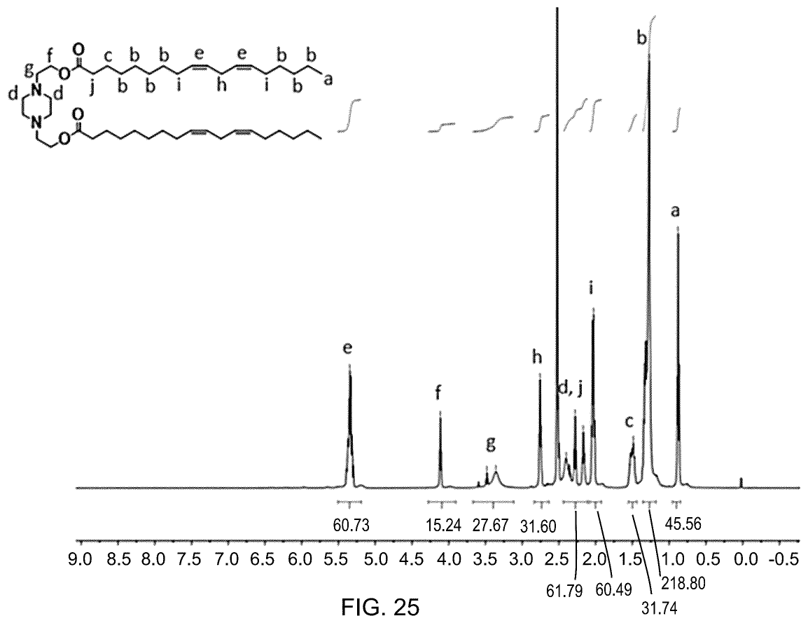

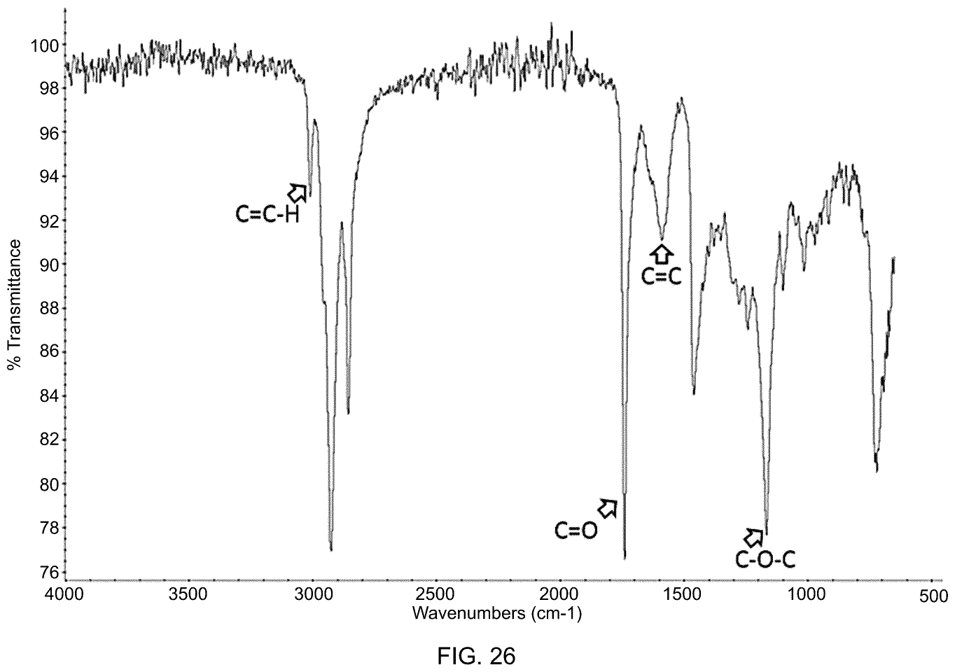

An ionizable cationic lipid is generally composed of three parts: (1) hydrophilic headgroups containing one or multiple ionizable amines for condensing negatively charged mRNA; (2) hydrophobic hydrocarbon chains capable of promoting self-assembly and phospholipid membrane fusion; (3) degradable ester linkers connecting the headgroups with hydrocarbon chains to potentially lower systemic cytotoxicity (FIG. 2) [10,11,28]. Herein, a new library of ionizable cationic lipids is designed, where the ionizable amine headgroups originated from different amino alcohols and the hydrocarbon lipid chains were derived from commercially available lipid acids. Through CALB enzyme-assisted reaction, the hydroxyl groups were reacted with carboxylic acids via one-step high-efficiency esterification (FIG. 1) [23] and an 18*8 library of lipid-like materials was synthesized by varying amino alcohols and lipid acids (FIG. 3 and FIG. 5). Preliminarily, these lipid-like materials with DOPE, cholesterol, and DMG-PEG were fabricated at a molar ratio of 50:10:38.5:1.5 which was the most widely used formulation [9, 10] to form LNPs and delivered luciferase encoded mRNA (mLuc) in vitro to generate a luciferase expression heat map of lipid-like materials (FIG. 4). The top-performing ionizable cationic lipid was screened and termed as AA3-DLin which was chemically composed of 1,4-Bis(2-hydroxyethyl) piperazine amine headgroups connected with two linoleic lipids by ester linkers as shown in the structure (FIGS. 1, 2). The AA3-DLin lipids were successfully synthesized by high-efficiency CALB-mediated catalytic esterification and characterized by electrospray ionization (ESI) mass spectrometry which showed a strong, clear and single peak denoting AA3-DLin with molecular weight (MW) of 699 and up to 96% purity (FIG. 24). Furthermore, nuclear magnetic resonance (NMR) spectroscopy and Fourier-transform infrared spectroscopy (FTIR) revealed the chemical structure of AA3-DLin accordingly (FIGS. 25-26). In one or more embodiments, the ionizable cationic lipid molecules comprise a molecular weight in a range of from 200 to 2000 Daltons, including all values and subranges therebetween.

FIGS. 1 to 4. Synthesis and screening of ionizable cationic lipids. FIG. 1 shows a chemical synthesis route of AA3-DLin through CALB enzyme-assisted esterification according to one or more embodiments to prepare amino alcohol mediated ionizable cationic lipid compounds. In one or more embodiments, the reaction of amino alcohol and lipid acid is conducted at 60° C. for 48 hours in the presence of an enzyme, e.g., CALB. FIG. 2 illustrates a schematic view of an ionizable cationic lipid 1 comprising an amino alcohol mediated ionizable cationic lipid compound, according to one or more embodiments. An amine head 2 supplied by the amino alcohol is attached by an ester linker 3 to lipid chains 4. FIG. 2 also illustrates a schematic view of a lipid nanoparticle 10. The therapeutic lipid nanoparticle 10 comprises an LNP fabricated by using one or more amino alcohol mediated ionizable cationic lipid compounds, with other lipid components, which assemble to form an outer shell 12 encapsulating a primary core, and inner layers 14. In one or more embodiments, a therapeutic lipid nanoparticle would include a nucleic acid-based agent, such as mRNA. The outer shell 12 and the inner layers 14 independently comprise an assembly of amino alcohol mediated ionizable cationic lipid compounds, with phospholipids, cholesterol and PEGylated lipids.

FIG. 3 shows chemical structures of amino alcohols and lipid acids used in the synthesis of lipid-like materials according to one or more embodiments. The amino alcohols are: (AA1) triethanolamine; (AA2)N-methyldiethanolamine; (AA3) 1,4-Bis(2-hydroxyethyl)piperazine; (AA4)N-butyl di ethanol amine; (AA5) 1-(2-hydroxyethyl)piperazine; (AA6) 1-(2-hydroxyethyl)piperidine; (AA7) 4-piperidineethanol; (AA8) 2-(methylamino)ethanol; (AA9) 1-amino-4-(2-hydroxyethyl)piperazine; (AA10) ethylenediamine; (AA11) 1-piperazinepropanol; (AA12) 4(5)-(hydroxymethyl)imidazole; (AA13) 2-Piperidineethanol; (AA14) N,N″-Bis(2-hydroxyethyl)ethylenediamine; (AA15) 2-(1-Methyl-1H-imidazol-2-yl)-ethanol; (AA16)N-(2-hydroxyethyl)ethylenedi amine; (AA17) 1-(3-hydroxypropyl)-1H-imidazole; and (AA18) triisopropanolamine. The Lipid Acid Library includes C4-C26, for example: octanoic acid (C8), decanoic acid (C10), dodecanoic acid (C12), myristic acid (C14), palmitic acid (C16), stearic acid (C18), oleic acid (C18:1(9)), and linoleic acid (C18:2(9, 12)). FIG. 4 shows luciferase expression heat map of mLuc-loaded LNPs. The LNPs were prepared with different lipid-like materials where the x-axis represents different amino alcohols and the y-axis represents different lipid acids used for synthesis. All the LNPs were formulated with ionizable cationic lipid, DOPE, cholesterol, and DMG-PEG at a molar ratio of 50:10:38.5:1.5 and a 1:20 weight ratio of mRNA to ionizable lipid (wt./wt.). All the transfection experiments were repeated in triplicate and the mean values were used to generate the heat map.

FIG. 5. The chemical structures of reaction ingredients for amino alcohol mediated ionizable cationic lipid compounds. According to one or more embodiments, R represents 8 different lipid acid aliphatic chains of the acids shown in FIG. 3 for grafting on 18 different amino alcohols.

Screening of AA3-DLin LNP Formulation by Orthogonal Design of Experiment

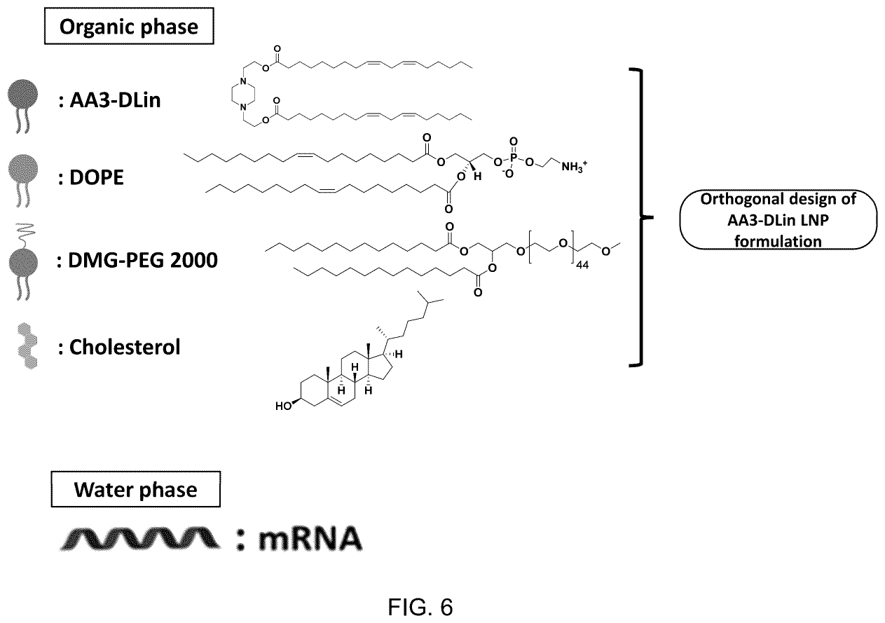

Without intending to be bound by theory, delivery efficacy of mRNA s not solely determined by individual components of the ionizable cationic lipid; molar ratio of among the four components in LNP formulation can also exert an effect. An orthogonal design of experiment (DoE) methodology was carried out to analyze various molar ratios of AA3-DLin, DOPE, cholesterol, and DMG-PEG (FIG. 6) [22,27,29]. The orthogonal design was used to screen 256 (4⁴) possible LNP formulations by combining four excipients of AA3-DLin, DOPE, cholesterol, and DMG-PEG with 16 formulations and evaluate four components with only 16 typical combinations. For the first-round of experiments (Library A), the relative molar ratios of the four components in the LNP formulation, including the determinate molar ratio of each component and the weight ratio of AA3-DLin to mRNA, are described in Table 1.

TABLE 1
Library A: orthogonal array table L₁₆ (4⁴) (molar ratio)

| | AA3- | | | | AA3-DLin/mRNA |
|---|---|---|---|---|---|
| Name | Dlin | DOPE | Cholesterol | DMG-PEG | (wt/wt) |
| A1 | 20 | 10 | 20 | 0.5 | 20 |
| A2 | 20 | 20 | 25 | 1 | 20 |
| A3 | 20 | 30 | 30 | 2.5 | 20 |
| A4 | 20 | 40 | 35 | 5 | 20 |
| A5 | 30 | 10 | 25 | 2.5 | 20 |
| A6 | 30 | 20 | 20 | 5 | 20 |
| A7 | 30 | 30 | 35 | 0.5 | 20 |
| A8 | 30 | 40 | 30 | 1 | 20 |
| A9 | 40 | 10 | 30 | 5 | 20 |
| A10 | 40 | 20 | 35 | 2.5 | 20 |
| A11 | 40 | 30 | 20 | 1 | 20 |
| A12 | 40 | 40 | 25 | 0.5 | 20 |
| A13 | 50 | 10 | 35 | 1 | 20 |
| A14 | 50 | 20 | 30 | 0.5 | 20 |
| A15 | 50 | 30 | 25 | 5 | 20 |
| A16 | 50 | 40 | 20 | 2.5 | 20 |

Briefly, formulations were prepared with the following ratios of AA3-DLin (20 to 50):DOPE (10 to 40):cholesterol (20 to 35):DMG-PEG (0.5-5), and a fixed 1:20 (wt./wt.) weight ratio of mRNA to AA3-DLin.

To further screen the formulation, a second round of experiments (Library B) was conducted where the relative molar ratios were fine-tuned as described in Table 2.

TABLE 2
Library B: orthogonal array table L₁₆ (4⁴) (molar ratio)

| | AA3- | | | | AA3-DLin/mRNA |
|---|---|---|---|---|---|
| Name | Dlin | DOPE | Cholesterol | DMG-PEG | (wt/wt) |
| B1 | 5 | 10 | 15 | 0 | 20 |
| B2 | 5 | 15 | 20 | 0.5 | 20 |
| B3 | 5 | 20 | 25 | 1 | 20 |
| B4 | 5 | 25 | 30 | 2 | 20 |
| B5 | 10 | 10 | 20 | 1 | 20 |
| B6 | 10 | 15 | 15 | 2 | 20 |
| B7 | 10 | 20 | 30 | 0 | 20 |
| B8 | 10 | 25 | 25 | 0.5 | 20 |
| B9 | 15 | 10 | 25 | 2 | 20 |
| B10 | 15 | 15 | 30 | 1 | 20 |
| B11 | 15 | 20 | 15 | 0.5 | 20 |
| B12 | 15 | 25 | 20 | 0 | 20 |
| B13 | 20 | 10 | 30 | 0.5 | 20 |
| B14 | 20 | 15 | 25 | 0 | 20 |
| B15 | 20 | 20 | 20 | 2 | 20 |
| B16 | 20 | 25 | 15 | 1 | 20 |

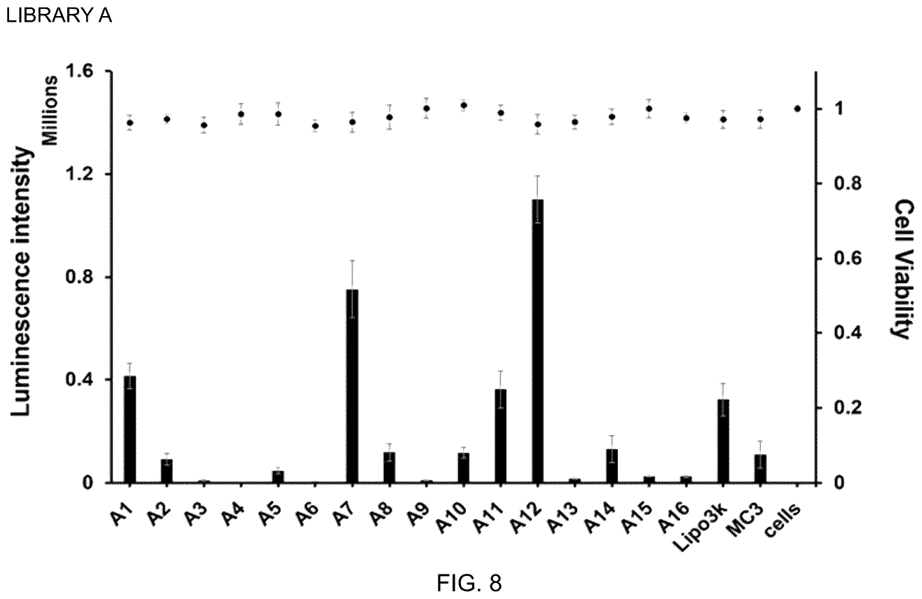

Briefly, formulations were prepared with the following ratios of AA3-DLin (5 to 20):DOPE (10 to 25):cholesterol (15 to 30):and DMG-PEG (0-2) with a fixed 1:20 (wt./wt.) ratio of mRNA to AA3-DLin. mLuc was used as a reporter gene to evaluate the transfection efficacy of LNP formulations on Hek 293 cells in vitro with the FDA-approved MC3[9] and commercial lipofectamine 3000 as control groups. Summarized by Table 1 and FIG. 8, increasing a portion of DMG-PEG dramatically lowers the transfection efficacy of AA3-DLin LNP in vitro. However, the DMG-PEG is also desirable for LNP transfection in vivo by preventing immune system detection of LNPs [7]. Therefore, to enhance AA3-DLin LNP transfection efficacy both in vitro and in vivo, the molar ratio of DMG-PEG was determined to be 0.5 in the AA3-DLin LNP formulation. Additionally, different from the FDA-approved siRNA-LNP formulations that frequently require a high portion of ionizable cationic lipid and a low portion of phospholipids [9, 10], we found that a high portion of both AA3-DLin and DOPE enhanced the mRNA transfection efficacy in vitro. Without intending to be bound by theory, it is thought that the phospholipids facilitate LNP fusion into cells [6]. Consequently, through high-throughput screening by Bright-Glo luciferase assay, the A12 formulation with a molar ratio of 40:40:25:0.5 (AA3-DLin:DOPE:Cholesterol:DMG-PEG) was determined to be a desirable formulation of AA3-DLin LNPs which showed a 6- and 3-fold higher transfection efficacy than MC3 and lipofectamine 3000, respectively. Notably, no cytotoxicity was observed for any of the AA3-DLin LNP formulations, which demonstrated the excellent biosafety properties of AA3-DLin LNPs.

FIGS. 6-9. Orthogonal design of experiment methodology for screening the optimal formulation of AA3-DLin LNP. FIG. 6 is a schematic view illustrating 4 components of AA3-DLin LNPs for Design of Experiment (DoE) calculation. The organic phase contains AA3-DLin, DOPE, cholesterol, and DMG-PEG at a molar ratio of according to one or more embodiments. The water phase contains an mRNA of interest (in NaOAc buffer) according to one or more embodiments. Two L₁₆ (4⁴) orthogonal tables were employed to guide the preparation of formulations.

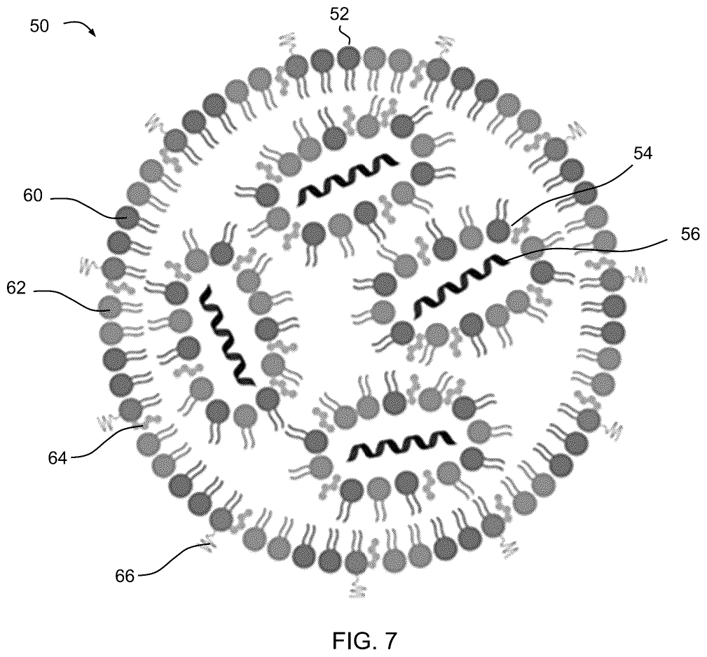

FIG. 7 is a schematic view of a therapeutic lipid nanoparticle comprising a lipid nanoparticle (LNP) encapsulating a nucleic acid agent according to one or more embodiments. In this embodiment, the therapeutic lipid nanoparticle 50 comprises an LNP fabricated by using one or more an amino alcohol mediated ionizable cationic lipid compounds, namely AA3-DLin, with other lipid components (DOPE, cholesterol, DMG-PEG 2000), which assemble to form an outer shell 52, and inner layers 54. The outer shell 52 and the inner layers 54 independently comprise an assembly of the amino alcohol mediated ionizable cationic lipid compound with the other lipid components, which includes therein a nucleic acid-based agent 56 such as mRNA. In one or more embodiments, some or all of the nucleic acid-based agent 56 is within one or more of the inner layer 54 structures. The outer shell 52 and the inner layers 54 independently comprise a random assembly of the amino alcohol mediated ionizable cationic lipid compound named “AA3-DLin” which is synthesized via esterification of 1,4-Bis(2-hydroxyethyl) piperazine and linoleic acid 60, DOPE 62, cholesterol 64, and DMG-PEG 2000 66.

Table 1 shows details of LNP formulations in Library A for first-round experiments, including the determinate molar ratio of each component and the weight ratio of AA3-DLin to mRNA. FIG. 8 shows the transfection efficacy and cell viability of different AA3-DLin LNP formulations in Library A (as well as lipofectamine 3000 and MC3), which were evaluated on Hek 293 cells.

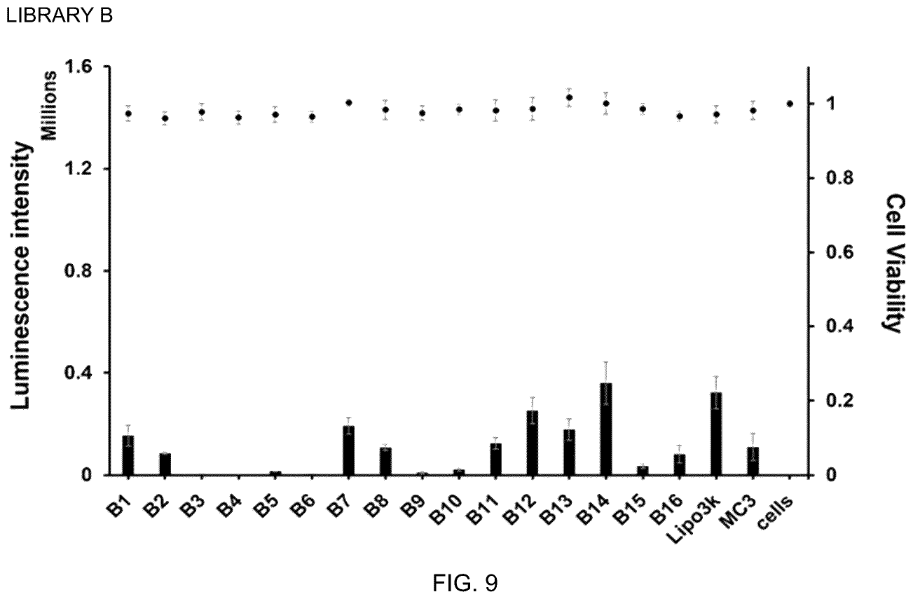

Table 2 shows details of LNP formulations in Library B for second-round experiments. FIG. 9 shows the transfection efficacy and cell viability of LNP formulations in Library B, which were evaluated on Hek 293 cells. Cell viability data were normalized to control groups. All experiments were repeated in triplicate to generate results and data are shown as mean±SD.

Microfluidic Fabrication and Characterization of LNPs

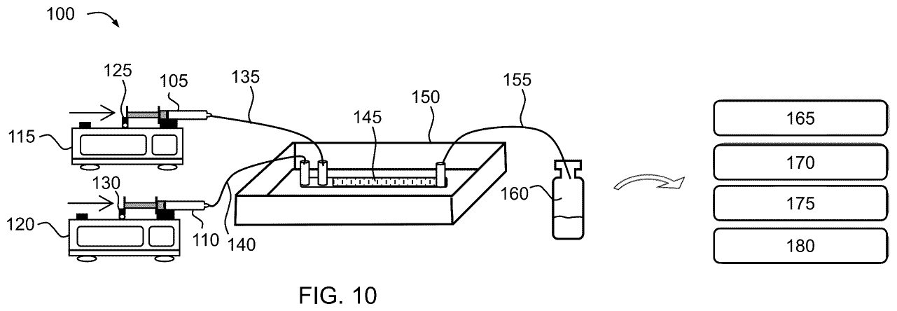

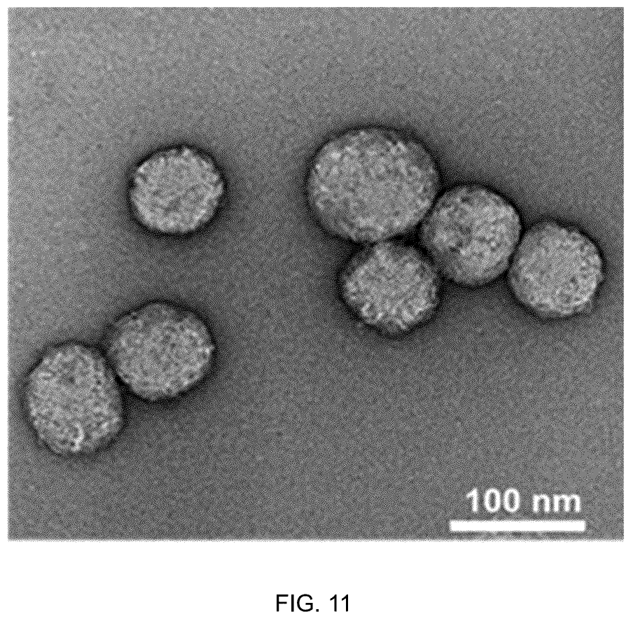

FIGS. 10 to 13. Fabrication of AA3-DLin LNPs using rapid microfluidic mixing. FIG. 10 shows a schematic illustration of LNP fabrication through microfluidic mixing. FIG. 11 shows the TEM image of AA3-DLin LNPs stained by uranyl acetate solution (scale bar: 100 nm).

Table 3 summarizes the characterizations of AA3-DLin LNPs fabricated by a microfluidic device with different syringe pump rates.

TABLE 3
Characterizations of AA3-DLin LNPs

| | | | | | Zeta | Encapsulation |
|---|---|---|---|---|---|---|
| Water phase | Organic phase | W/O | Size | | potential | efficacy |
| (μL/min) | (μL/min) | ratio | (nm) | PDI | (mV) | (%) |
| 300 | 100 | 3:1 | 156.5 ± 11.8 | 0.168 ± 0.032 | −1.2 ± 2.1 | 91.8 ± 3.6 |
| 900 | 300 | 3:1 | 114.6 ± 5.3 | 0.156 ± 0.025 | −2.6 ± 3.5 | 93.2 ± 1.5 |
| 1500 | 500 | 3:1 | 96.2 ± 4.1 | 0.132 ± 0.016 | −2.2 ± 2.2 | 95.7 ± 0.8 |
| 2400 | 800 | 3:1 | 89.7 ± 3.8 | 0.128 ± 0.012 | −1.8 ± 2.6 | 95.5 ± 1.2 |

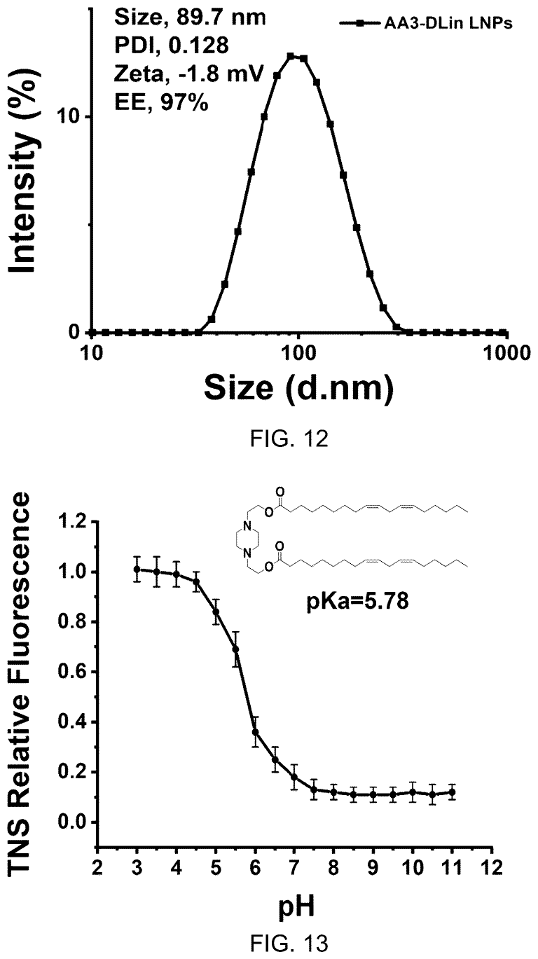

FIG. 12 shows the characterization summary of LNPs prepared under high pump rates. FIG. 13 shows pKa value of AA3-DLin LNPs measured by TNS fluorescence titration. pKa was defined as the pH at half-maximal fluorescence intensity. All experiments were repeated in triplicate to generate results and data are shown as mean±SD. In addition to components of the ionizable cationic lipid and the LNP formulation ratios, the nanoparticle size also affects the transfection efficacy and vaccine immunogenicity [30]. Microfluidic-chip device (FIG. 10) 100 facilitates LNP fabrication methods to obtain uniform nanoparticle in laboratory[31]. AA3-DLin, DOPE, cholesterol, and DMG-PEG were dissolved separately in ethanol at 10 mg/mL and mixed at a molar ratio of 40:40:25:0.5, respectively to obtain an organic phase of the A12 formulation of AA3-DLin LNPs. Additionally, the mRNA water phase was prepared by dissolving the mRNA of interest in NaOAc buffer (pH 5.0, 25 mM) (FIG. 6). The volume ratio of the LNP organic phase to the mRNA water phase was fixed at 1:3 (v/v) and separately loaded into different syringes 105 and 110, which were positioned on respective weigh cells 115 and 120. The LNP production process is detailed in the following operations: (1) two material phases (organic and water) were pumped, e.g., by respective pumps 125, 130 through respective tubing/piping 135, 140 into microfluidic channels 145 located in a base 150, and rapidly mixed in the microfluidic channels 145 to obtain uniform LNPs, which were collected in a vessel 160 through an outlet 155; (2) at operation 165 the resulting LNPs were incubated at RT (room temperature, e.g., 20° C.±5° C.) for 30 min allowing for mRNA encapsulation [30]; (3) at operation 170 the LNPs were further dialyzed against 1×PBS for 2 hours at RT to remove the ethanol. Then the LNPs were ready for storage at operation 175 and for characterization at operation 180, and administration in animal studies (FIG. 10). In the microfluidic device 100, the two phases were rapidly pumped and thoroughly mixed to obtain uniform LNPs through the micro-sized channels 145 where the positively charged head group of the AA3-DLin lipid can electrostatically interact with negatively charged mRNAs in an acidic buffer and allow mRNA encapsulation inside LNPs (FIG. 7). Notably, we observed that the LNP size can be controlled by adjusting the pump rates. By increasing the pump rate to 2400 μL/min for the water phase and 800 μL/min for the organic phase, the LNPs obtained (FIG. 11) were (for example) 89 nm in size (particle diameter) with 0.128 PDI (polydispersity index) (Table 3 and FIG. 12), as measured by a Zeta Sizer dynamic light-scattering (DLS) detector discussed below. At low pump rates, the resulting size of LNPs ranged from 114.6 to 156.5 nm (Table 3). Additionally, adjusting the pump rates did not distinctly affect the zeta potential and encapsulation efficacy of the LNPs exemplified by the fact that all the obtained LNPs have a neutral zeta potential and more than 90% encapsulation efficacy (Table 3). The TEM images clearly showed that the A12 formulation of AA3-DLin LNPs have a uniform compact spherical shape with a diameter around 90 nm which is consistent with the DLS result. Moreover, the pKa value is a characteristic of the ionizable cationic lipid which signifies the pH when the LNPs are 50% protonated, indicating the LNPs' capability to release the encapsulated mRNA payload into the cytoplasm in the acidic endosomal environment [9,32,33]. The pKa of AA3-DLin LNP was determined by measuring the fluorescence of 2-(p-toluidino)-6-napthalene sulfonic acid (TNS). The formulated AA3-DLin LNPs were titrated from a pH range of 3.0 to 11.0 in increments of 0.5 pH units and a fluorescence titration “S” curve was generated. After a curve-fit analysis, the pKa of AA3-DLin LNPs was determined to be 5.78 which was the pH value at 50% of maximum fluorescence (FIG. 13).

A Comprehensive Evaluation of AA3-DLin LNPs In Vitro and In Vivo

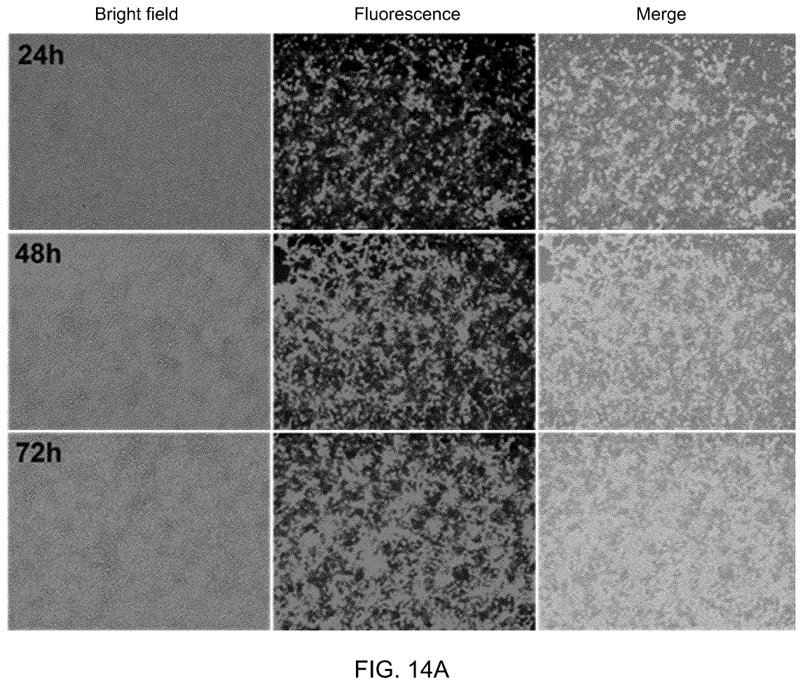

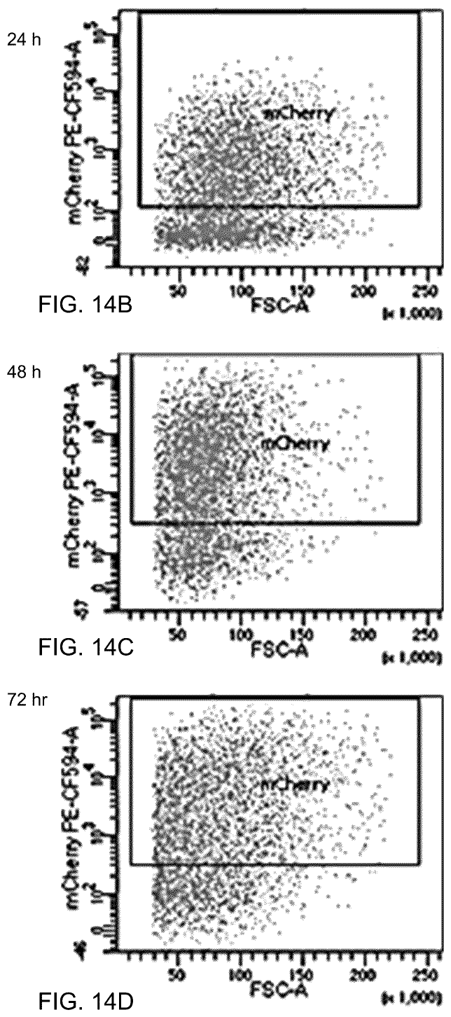

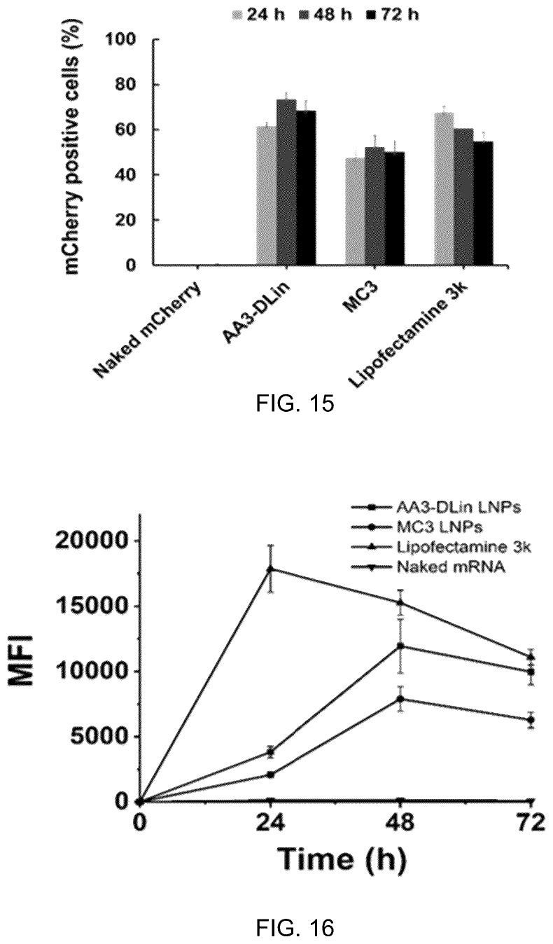

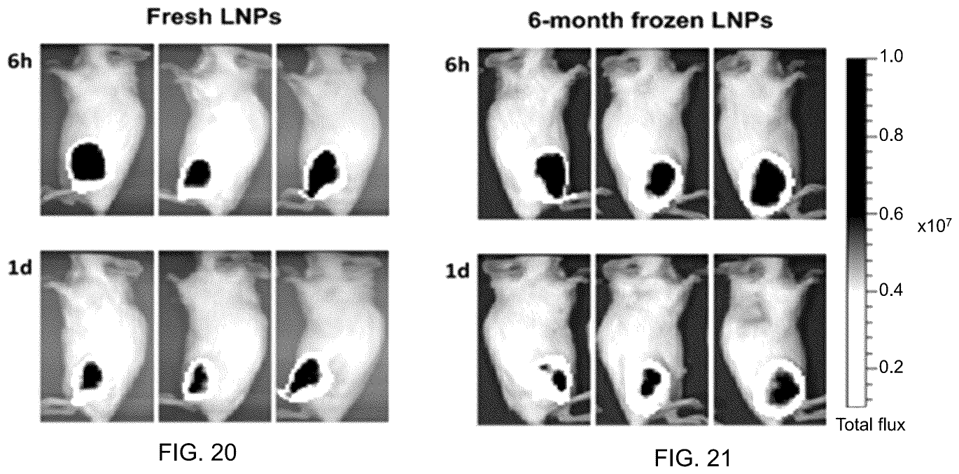

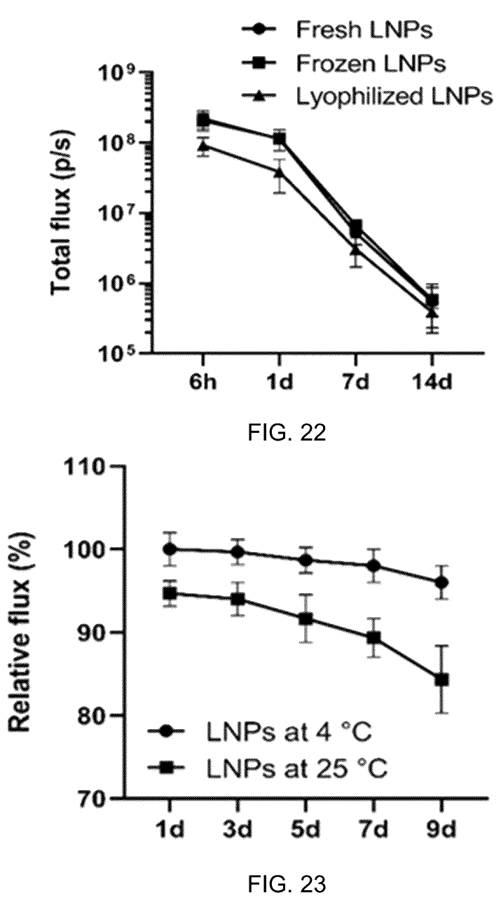

A comprehensive evaluation of AA3-DLin LNPs was carried out in terms of transfection efficacy in vitro and in vivo where the LNPs were prepared by A12 formulation (40:40:25:0.5) and fabricated by the microfluidic device under the optimized high-speed pump rate with the mRNA of interest. First, the in vitro transfection efficacy of AA3-DLin LNPs were evaluated by delivering mCherry encoded mRNA (mCherry-LNPs) on Hek 293 cells. The mCherry-expressing cells were visualized by a fluorescence microscope (FIG. 14A) and analyzed by flow cytometry (FIGS. 14B-14D). The flow cytometry results showed the population of mCherry transfected cells reached around 78% at 48 h post-transfection whereas the FDA-approved MC3 LNPs and lipofectamine 3000 groups showed the percentage of mCherry positive cells were 58% and 62%, respectively (FIG. 15). Additionally, the mean fluorescence intensity (MFI) of the mCherry positive cells showed that the AA3-DLin LNPs outperformed the MC3 LNPs (FIG. 16). The agarose gel electrophoresis results demonstrated that the fresh and long-term stored AA3-DLin LNPs bound mRNAs efficiently at both 1:10 and 1:20 weight ratio of mRNA to AA3-DLin lipid. Next, we investigated the AA3-DLin LNP transfection efficacy in vivo by delivering luciferase encoded mRNA (mLuc-LNPs). The mLuc-LNPs were intramuscularly injected into BALB/c mice to mimic typical clinical vaccination approaches. Remarkably, the AA3-DLin LNPs efficiently delivered the mRNA in vivo and generated strong luciferase expression at a scale of 2.65*10⁸ total flux (photons/seconds) at 6 hours post-injection and the luciferase expression lasted for at least 2 weeks (FIGS. 20, 22).

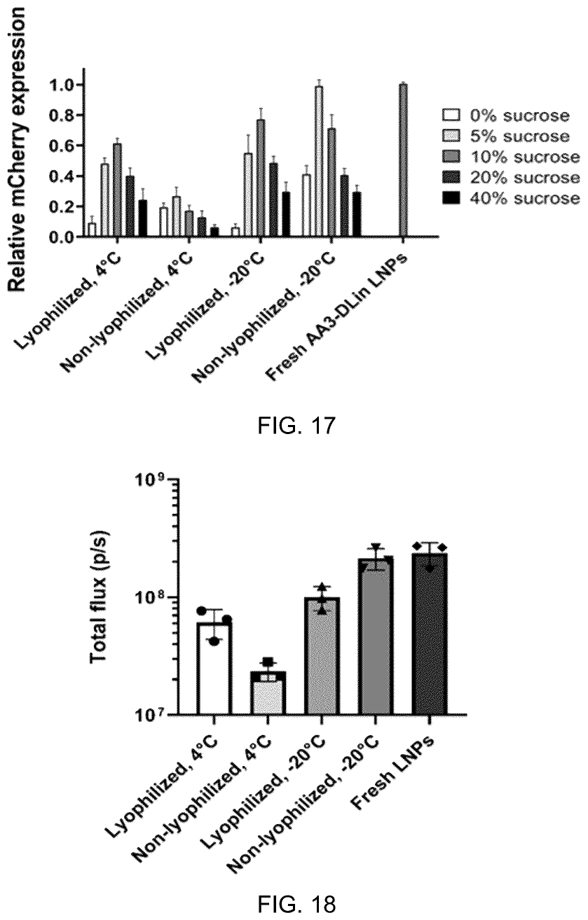

We then explored the long-term storage capability and thermostability of AA3-DLin LNPs under different storage conditions and selected sucrose as the cryoprotectant to stabilize the LNP structure during storage [34,35], Sucrose solution was added to LNPs with final concentrations of 0-40 wt. % and the resulting LNPs were further lyophilized as a powder or stored at either 4° C. or −20° C. with a timeline of 12 months. The transfection efficacy of stored LNPs was characterized in vitro and in vivo with the fresh LNPs as the positive control group. After 12 months of storage at −20° C., the frozen mCherry-LNPs with wt. % sucrose showed no loss of transfection efficacy compared with the fresh LNPs. However, after 12 months of storage at 4° C., the mCherry-LNPs solution exhibited a dramatic decrease in transfection efficacy, probably due to the hydrolysis-mediated degradation of LNPs and mRNAs. For the lyophilized mCherry-LNPs, the best sucrose concentration was determined to be 10 wt. % after analysis and the lyophilized LNPs showed a moderate decrease in transfection efficacy compared to fresh LNP after 6-month storage at −° C. (FIG. 17). Notably, we observed that the sucrose significantly affected the transfection efficacy of lyophilized LNPs [35], probably due to the sucrose moiety facilitating the resuspension of the LNP lyophilized powders into homogeneous solution 35 (FIG. 17). Furthermore, we characterized the transfection efficacy of mLuc-LNPs stored for 12 months in BALB/c mice where the frozen LNPs and lyophilized LNPs were fixed with 5 wt. % and wt. % sucrose, respectively. Consistent with the cellular experiments, the frozen mLuc-LNPs (5 wt. % sucrose) showed no reduction in transfection efficacy compared with fresh LNPs after 12 months of storage at −20° C. (FIGS. 21, 18) and still generated strong luciferase expression ˜2.51*10⁸ total flux (p/s) at 6 h post-administration, which lasted for 2 weeks (FIG. 22). However, the lyophilized LNPs (10 wt. % sucrose) showed a loss of transfection efficacy, from 2.65*10⁸ to 9.82*10⁷ total flux (p/s) in vivo over 12 months of storage at −20° C. (FIG. 18). Without intending to be bound by theory, it is hypothesized that the oxidization of unsaturated lipids in the lyophilized LNPs leads to the loss of transfection efficacy during storage since the degree of unsaturation of lipids has a positive effect on LNP transfection efficacy [36].

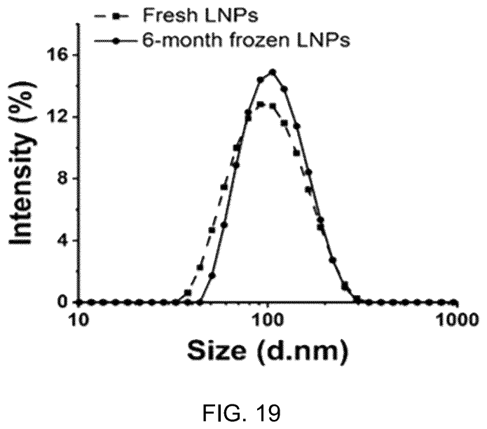

Notably, the LNPs stored frozen for 12 months represented remarkable stability where the particle size slightly changed from 89 nm to 92 nm and no obvious change in zeta potential compared to fresh LNPs (FIG. 19). The stability test results showed the AA3-DLin LNPs maintained in vivo delivery efficacy at 4° C. for at least one week (FIG. 23). The comprehensive studies demonstrated that AA3-DLin LNPs showed outstanding mRNA delivery efficacy in vitro and in vivo, and showed excellent thermostability at 4° C. as well as long-term storage capability at −20° C. without loss of mRNA delivery efficacy.

FIGS. 14A-14D to 23. A comprehensive evaluation of AA3-DLin LNPs in vitro and in vivo. FIG. 14A shows fluorescence imaging and FIGS. 14B-14D show flow cytometry results of Hek 293 cells transfected by mCherry-LNPs at different time points. FIG. 15 shows a summary of mCherry positive cell population transfected by different formulations. FIG. 16 shows mean fluorescence intensity (MFI) of mCherry positive cells according to flow cytometry data. FIG. 17 shows transfection evaluation of 6-month stored mCherry-LNPs in vitro, the data were summarized by flow cytometry and normalized to fresh LNPs. FIG. 18 shows summary of luciferase expression generated by different long-term stored mLuc-LNP formulations in vivo, the luciferase expression was recorded after 6 hours post-administration. FIG. 19 shows the comparison of particle size and distribution between fresh LNPs and 6-month frozen stored LNPs. FIGS. 20 and 21 show in vivo bioluminescence (total flux) images of BALB/c mice after administration of fresh mLuc-LNPs and 6-month frozen mLuc-LNPs (n=3), respectively, at specific time points. FIG. 22, shows in vivo transfection efficacy comparison between fresh mLuc-LNPs, 6-month frozen mLuc-LNPs, and 6-month lyophilized mLuc-LNPs. The luciferase expression was recorded for 2 weeks. FIG. 23 shows the mLuc-LNPs were stored at 4° C. or 25° C. (room temperature) for 1, 3, 5, 7, and 9 days before injecting to mice. The luciferase expression was recorded after 6 hours post-administration and normalized to fresh mLuc-LNPs. All experiments were repeated in triplicate and the data were represented as means±SD.

In summary, the present inventors reported the design, synthesis and application of a new class of amino alcohol mediated ionizable cationic lipid compounds to develop LNPs for gene delivery. The transfection studies have solidly proven the potential of the AA3-DLin LNPs to be an effective and robust mRNA delivery platform with excellent thermostability and long-term storage capability without ultracold conditions, lending itself for adaption to limitless uses in mRNA-LNP delivery applications.

### Materials and Methods

Materials

All amino alcohol reagents and lipid acids such as octanoic acid (C8), decanoic acid (C10), dodecanoic acid (C12), myristic acid (C14), palmitic acid (C16), stearic acid (C18), oleic acid (C18:1(9)), and linoleic acid (C18:2(9, 12)) were purchased from Sigma-Aldrich (St. Louis, MO, USA). (6Z,9Z,28Z,31Z)-Heptatriaconta-6,9,28,31-tetraen-19-yl 4-(dimethylamino) butanoate (DLin-MC3-DMA) were obtained from Ambeed, Inc. (IL, USA). 1,2-dioleoyl-sn-gly cero-3-phospho ethanol amine (DOPE), 1,2-di stearoyl-sn-gly cero-3-phosphocholine (DSPC) and 1,2-dimyristoyl-rac-glycero-3-methoxypolyethylene glycol-2000 (DMG-PEG 2000) were purchased from Avanti Polar Lipids, Inc. (AL, USA). Cholesterol, Cell Counting Kit-8 (CCK-8), Sodium acetate buffer (NaOAc, pH 5.0), Candida Antarctica Lipase B (CALB), anhydrous Magnesium sulfate and Pur-A-Lyzer Midi Dialysis Kits (MWCO 3.5 kDa) were obtained from Sigma-Aldrich (St. Louis, MO, USA). Dulbecco's Modified Eagle Medium (DMEM, with 4.5 g/L D-Glucose, L-Glutamine and 110 mg/L Sodium Pyruvate), Opti-MEM reduced Serum Medium, Heat-Inactivated Fetal Bovine Serum, Penicillin-Streptomycin and 0.25% Trypsin-EDTA (1×) were purchased from Gibco (Paisley, UK). Lipofectamine 3000 Reagent was obtained from Invitrogen (Carlsbad, CA, USA). Firefly luciferase encoded mRNA (mLuc) and mCherry encoded mRNA (mCherry) were purchased from TriLink Biotechnologies, Inc. (San Diego, CA). The full-length spike protein encoded mRNA was obtained from System Biosciences. Bright-Glo luciferase assay substrate was from Promega (Madison, WI). D-Luciferin potassium salt was obtained from PerkinElmer Inc. All lab supplies and analytical grade reagents were from VWR (Radnor, PA, USA) and Sigma-Aldrich (St. Louis, MO, USA).

Ionizable Cationic Lipid Library Synthesis

The ionizable cationic lipids were synthesized through a one-step enzymatic esterification between hydroxyl groups (—OH) of amino alcohol and carboxylic acid groups (—COOH) of lipid acids using CALB-immobilized on acrylic resin as a catalyst [23]. Briefly, amino alcohol was dissolved in THF in a 100 mL two-neck round bottom flask followed by the addition of lipid acid with a molar ratio of hydroxyl group/carboxylic acid group of 1:2 and 0.5 g CALB as esterification catalyst. The mixture was stirred, and the reactions took place at 60° C. under nitrogen protection for 48 hours. After 48 hours reaction, the CALB was removed by centrifuge, the supernatant was collected and purified by saturated NaHCO₃ solution to neutralize the excess lipid acid. Then the resultant was extracted by excess ethyl acetate following drying with anhydrous MgSO₄. A clear lipid ethyl acetate solution was obtained after removing MgSO₄ by centrifuge and the lipid solution was concentrated by rotatory evaporating to remove the ethyl acetate and the resulting lipids were dried in vacuum for 48 h until use. The purified lipid product was confirmed by ¹H NMR spectrometry (Bruker 500 MHz NMR spectrometer) and High Resolution Orbitrap Q Exactive LC/MS spectrometry (Thermo Fisher Scientific), and the FTIR spectra were collected using a Nicolet IS-10 (Thermo Fisher Scientific).

High-Throughput Screening of Ionizable Cationic Lipid Library In Vitro

The ionizable cationic lipid candidates were synthesized through CALB enzyme-assisted esterification between the amino alcohol (AA) library and lipid acid library. The lipids were named in the form of AA #-lipid. In all cases, “#” indicates an assigned number of an amino alcohol as shown in FIG. 3 (“Amino Alcohol Library”) and FIG. 5, “lipid” indicates the different hydrophobic carbon tails such as C8 and Lin in FIG. 3 (“Lipid Acid Library”). In the preliminary screening of the lipid acid library, the ionizable lipids, DOPE, cholesterol, and DMG-PEG were dissolved in ethanol at the concentration of 10 mg/mL and the luciferase encoded mRNA (mLuc) was diluted in NaOAc buffer (25 mM, pH 5.0). The mLuc loaded LNPs were formulated via pipetting the mRNA NaOAc solution to lipid ethanol mixtures at a volume ratio of 3:1 (NaOAc:ethanol, v/v) and incubated for 30 min at room temperature before in vitro screening with a previously reported molar ratio of 50:10:38.5:1.5⁹ and a fixed weight ratio of 1:20 (mRNA:ionizable lipid, wt./wt.). These different mLuc (0.1 μg) loaded LNPs were used to transfect Hek 293 cells seeded in a 96-well plate, and the LNP mediated mRNA transfection efficacy was determined by the luciferase expression intensity which was analyzed by Bright-Glo luciferase assay according to the manufacturer's instruction and recorded by Tecan Infinite M200 Pro plate reader (Tecan) after 24 hour incubation.

Orthogonal Design of Experiment for Optimizing LNP Formulation

An orthogonal design of experiment (DoE) methodology was carried out to identify a desirable molar ratio of the four components in AA3-DLin LNP formulation according to an embodiment. Different AA3-DLin LNP formulations were prepared according to details in two orthogonal design tables L₁₆ (4⁴) termed Library A (Table 1) and Library B (Table 2) and use mLuc as a reporter gene. These mLuc (0.1 μg) loaded LNP formulations were used to transfect Hek 293 cells seeded in a 96-well plate, and the transfection efficacy was measured by Bright-Glo luciferase assay and recorded by plate reader after 24 hours post-transfection. The desirable molar ratio of AA3-DLin LNP formulation was determined to be A12 with a 40:40:25:0.5 molar ratio of AA3-DLin:DOPE:cholesterol:DMG-PEG and a fixed weight ratio of 1:20 (mRNA:AA3-DLin, wt./wt.) and used for all the further studies.

The MC3 LNPs were formulated with DSPC, cholesterol, and DMG-PEG 2000 at a molar ratio of 50:10:38.5:1.5 and a fixed weight ratio of 1:20 (mRNA:MC3, wt./wt.) according to previous report [9] and transfected cells with the same manner as described above. The lipofectamine 3000 was used according to the manufacturer's instructions. Additionally, the cell viability was tested by Cell Counting Kit-8 (CCK-8, Sigma-Aldrich) according to the manufacturer's instructions and the cell viability data was normalized to the value of the control group.

Fabrication of mRNA Loaded LNPs by Microfluidic Chip Device

A microfluidic chip device was applied to fabricate the LNPs with uniform particle size compared to mixing by pipetting. The microfluidic chip device used for AA3-DLin LNP fabrication was reported in the previous study [31]. The ethanol phase contained a mixture of AA3-DLin, DOPE, cholesterol, and DMG-PEG 2000 at a molar ratio of 40:40:25:0.5. The aqueous phase was prepared in 25 mM NaOAc buffer (pH 5.0) with either luciferase encoded mRNA (mLuc), mCherry encoded mRNA (mCherry) or antigen full-length spike encoded mRNA (mspike). The ethanol and aqueous phases were loaded in two different syringes at a volume ratio of 1:3 and a fixed mRNA/AA3-DLin weight ratio of 1:20 for in vitro and 1:10 for in vivo, respectively. The two phases were mixed in a microfluidic chip device using syringe pumps with pre-set pump rates. The resulting LNPs were subsequently incubated for 30 min at room temperature before dialysis against 1× PBS in a Pur-A-Lyzer Midi Dialysis Kit (MWCO 3.5 kDa) for 2 hours at 4° C. to remove ethanol. The fresh formulated LNPs could be concentrated or diluted on demands and used directly for in vitro and in vivo experiments. For the long-term storage studies of AA3-DLin LNPs, different concentration of sucrose solution (acting as cryoprotectant) was added and mixed thoroughly with the fresh LNPs. Freeze-drying process was carried out for the lyophilized LNP groups. Then the lyophilized LNP powders or LNP formulations were stored at either 4° C. or −20° C. for at least 12 months. The lyophilized LNP powders were resuspended thoroughly in 25 mM NaOAc buffer (pH 5.0) and the frozen LNP were thawed at room temperature before experiments.

LNP Characterizations and Morphology Analysis

The size (particle diameter), polydispersity index (PDI) and zeta potentials of AA3-DLin LNPs were measured in DI water using a Zeta Sizer dynamic light-scattering (DLS) detector (15-mW laser, incident beam of 676 nm; Malvern, UK) at 25° C. and at a scattering angle of 90°. The intensity-weighted mean value was recorded as the average of three measurements.

The mRNA encapsulation was determined by Quant-iT RiboGreen RNA Assay Kit (Invitrogen) according to the manufacturer's instructions. Briefly, the mRNA-loaded AA3-DLin LNP solution was incubated with Ribogreen, and the fluorescence intensity was measured to obtain the unencapsulated mRNA. Then, the AA3-DLin LNP solution was incubated with 2% Triton X-100 (Sigma-Aldrich) for 10 min to break apart the particles and release out mRNA, the fluorescence intensity was measured after incubating with Ribogreen as total mRNA [30,31,37]. The mRNA encapsulation efficiency (%) was calculated from the following equation:

$$mRNA ( encapsulation efficiency \% ) = \frac{( total mRNA - free mRNA )}{total mRNA} * 100 \%$$

The acid dissociation constant (pKa) was measured by 2-(p-toluidino)-6-napthalene sulfonic acid (TNS, Sigma-Aldrich) assay as reported before [9,27,37]. Briefly, mRNA-loaded AA3-DLin LNP formulation was added to a series of buffer containing 10 mM HEPES, 10 mM MES, 10 mM ammonium and 130 mM NaCl, where the pH ranged from 3.0 to 11.0. TNS was prepared as a 100 μM stock solution in DI water and incubated with the above solution for 5 min with slight shaking. The total volume of each sample was 100 μL in a 96-well plate with a final concentration of 5 μM TNS and 50 μM total lipid. The fluorescence intensity was measured using a Tecan plate reader with excitation and emission wavelengths of 321 nm and 445 nm and the data was normalized to the value of pH 3.0. The pKa was recorded as the pH at half-maximal fluorescence intensity.

The agarose gel electrophoresis assay was performed to investigate the binding efficiency of mRNA-loaded AA3-DLin LNP formulations. The LNPs were prepared with a weight ratio of mRNA to AA3-DLin as 1:10 and 1:20 for both fresh and stored LNPs. Then, 10 uL LNP samples were mixed with 2 uL RNA loading dye (New England Biolab) and loaded on a 1% agarose gel for 30 min at 110 V. The gel images were acquired using a ChemiDoc™ Gel Imaging System (Bio-Rad Laboratories).

The morphology of AA3-DLin LNPs were observed under a transmission electron microscope (JEM-F200 TEM, USA). Samples were prepared by placing 5 μL LNPs on a TEM grid and blotting away with filter paper after 1 min. The LNP sample was stained with 5 μL of 2% uranyl acetate solution for 30 s, then the uranyl acetate was removed by filter paper. The staining procedures were repeated another 2 times in the same manner. After staining, the samples were placed in the fume hood for 10 min drying and then directly observed using TEM.

Animals and Cells

All animal procedures were performed with ethical compliance and approval by the Animal Care and Use Committee at Rutgers-New Jersey Medical School. Female BALB/c mice (6-8 weeks) were supplied from the Jackson Laboratory and housed in Rutgers-New Jersey Medical School animal facility.

The Hek 293 cell line was kindly given by Dr. Lei Bu from NYU Langone Medical Center. The ACE2-293T stable cell line was a gift from Dr. Abraham Pinter and Dr. Dongfang Liu from Rutgers-New Jersey Medical School. The cells were cultured in high glucose Dulbecco's modified Eagle's medium (DMEM; Gibco Life Technologies, Carlsbad, CA) supplemented with 10% fetal bovine serum (FBS; Sigma-Aldrich), and 1% penicillin-streptomycin (Gibco Life Technologies, Carlsbad, CA). All cells were cultured at 37° C. in a humidified incubator with 5% CO₂.

Evaluation of LNP Transfection Efficacy by Fluorescence Microscopy and Flow Cytometry

The in vitro transfection efficacy of LNPs was also evaluated in the Hek 293 cell line by delivering mCherry encoded mRNA (mCherry-LNP) as a reporter gene. Briefly, 1×10⁵ Hek 293 cells were seeded into each well of 24-well plate with 0.5 mL complete culture medium and incubated at 37° C. overnight to allow cells attachment. The next day, fresh mCherry-LNPs or the long-term stored mCherry-LNPs were directly added into cell medium with a fixed 1 μg mCherry per well. The transfected mCherry positive cells were observed under All-in-One Fluorescence Microscope (BZ-X710, Keyence, Japan) at predetermined time points with brightfield, fluorescent and merged channels using 10×PanFluor lens (Nikon, Japan) (FIG. 14A). After observation and imaging, the cells were trypsinized by 0.25% Trypsin-EDTA (Sigma-Aldrich), followed by the addition of 0.5 mL 1×PBS and centrifuged to decant supernatant to get cell pellets. The cell pellets were resuspended in 0.5 mL PBS for flow cytometric analysis using a BD LSR II flow cytometer (BD Biosciences, San Jose, CA) and the data were analyzed using FACSDiva software (BD Biosciences, San Jose, CA) (FIGS. 14B-14D). Data were acquired using a 561 nm laser with a 610/20 BP filter for the detection of mCherry positive cells under a voltage of 350 V. At least 10,000 events were collected for each measurement.

LNPs Administration and Bioluminescence In Vivo

Female, 6-8 weeks old BALB/c mice (18-20 g) from Jackson Laboratory were used for the in vivo bioluminescence imaging study. ˜100 μL of fresh luciferase mRNA (10 μg, ˜0.5 mg/kg) encapsulated AA3-DLin LNPs (n=3) were injected into mice via intramuscular route at the hind leg. 10 μg luciferase mRNA diluted in 90 μL PBS (n=3) were injected intramuscularly into mice as control groups. For AA3-DLin LNP long-term storage studies, the lyophilized luciferase mRNA (10 μg, ˜0.5 mg/kg) encapsulated LNPs (n=3) (with wt. % sucrose) were resuspended in 100 μL NaOAc buffer for 30 min incubation at RT, and the frozen luciferase mRNA (10 μg, ˜0.5 mg/kg) encapsulated LNPs (n=3) (with 5 wt. % sucrose) were thawed at RT before mice injections with the same manners. For stability studies, the AA3-DLin LNPs (n=3) formulated with 5 wt. % sucrose were stored at either 4° C. or 25° C. (room temperature) for 1, 3, 5, 7 and 9 days before injecting into mice. At indicated time points, mice were injected with 150 μL D-Luciferin potassium salt (30 mg/mL, PerkinElmer) intraperitoneally and anesthetized in a ventilated anesthesia chamber with 1.5% isofluorane in oxygen. The luminescence signals were collected by IVIS spectrum instrument (IVIS-200, Xenogen, PerkinElmer) with an exposure time of 30 s after 15-20 minutes. Bioluminescence values were quantified by measuring photon flux (photons/second) in the region of interest using the In Vivo Imaging software provided by PerkinElmer.

### REFERENCES

1 Sahin, U. et al. COVID-19 vaccine BNT162b1 elicits human antibody and TH 1 T cell responses. Nature 586, 594-599 (2020).
2 Walsh, E. E. et al. Safety and immunogenicity of two RNA-based Covid-19 vaccine candidates. New England Journal of Medicine 383, 2439-2450 (2020).
3 Baden, L. R. et al. Efficacy and safety of the mRNA-1273 SARS-CoV-2 vaccine. New England Journal of Medicine 384, 403-416 (2021).
4 Widge, A. T. et al. Durability of responses after SARS-CoV-2 mRNA-1273 vaccination. New England Journal of Medicine 384, 80-82 (2021).
5 Ho, W. et al. Next-Generation Vaccines: Nanoparticle-Mediated DNA and mRNA Delivery. Advanced Healthcare Materials 10, 2001812 (2021).
6 Cullis, P. R. & Hope, M. J. Lipid nanoparticle systems for enabling gene therapies. Molecular Therapy 25, 1467-1475 (2017).
7 Hou, X., Zaks, T., Langer, R. & Dong, Y. Lipid nanoparticles for mRNA delivery. Nature Reviews Materials, 1-17 (2021).
8 Chaudhary, N., Weissman, D. & Whitehead, K. A. mRNA vaccines for infectious diseases: Principles, delivery and clinical translation. Nature Reviews Drug Discovery, 1-22 (2021).
9 Jayaraman, M. et al. Maximizing the potency of siRNA lipid nanoparticles for hepatic gene silencing in vivo. Angewandte Chemie 124, 8657-8661 (2012).
10 Semple, S. C. et al. Rational design of cationic lipids for siRNA delivery. Nature biotechnology 28, 172-176 (2010).
11 Miao, L., Zhang, Y. & Huang, L. mRNA vaccine for cancer immunotherapy. Molecular cancer 20, 1-23 (2021).
12 Qiu, M. et al. Lipid nanoparticle-mediated codelivery of Cas9 mRNA and single-guide RNA achieves liver-specific in vivo genome editing of Angptl3. Proceedings of the National Academy of Sciences 118 (2021).
13 Musunuru, K. et al. In vivo CRISPR base editing of PCSK9 durably lowers cholesterol in primates. Nature 593, 429-434 (2021).
14 Gillmore, J. D. et al. CRISPR-Cas9 in vivo gene editing for transthyretin amyloidosis. New England Journal of Medicine 385, 493-502 (2021).
15 Lin, Y-X. et al. Reactivation of the tumor suppressor PTEN by mRNA nanoparticles enhances antitumor immunity in preclinical models. Science Translational Medicine 13 (2021).
16 Hotz, C. et al. Local delivery of mRNA-encoded cytokines promotes antitumor immunity and tumor eradication across multiple preclinical tumor models. Science Translational Medicine 13, eabc7804 (2021).
17 Buschmann, M. D. et al. Nanomaterial delivery systems for mRNA vaccines. Vaccines 9, 65 (2021).
18 Sabnis, S. et al. A novel amino lipid series for mRNA delivery: improved endosomal escape and sustained pharmacology and safety in non-human primates. Molecular Therapy 26, 1509-1519 (2018).
19 Xu, X. et al. Enhancing tumor cell response to chemotherapy through nanoparticle-mediated codelivery of siRNA and cisplatin prodrug. Proceedings of the national academy of sciences 110, 18638-18643 (2013).
20 Love, K. T. et al. Lipid-like materials for low-dose, in vivo gene silencing. Proceedings of the National Academy of Sciences 107, 1864-1869 (2010).
21 Dong, Y. et al. Lipopeptide nanoparticles for potent and selective siRNA delivery in rodents and nonhuman primates. Proceedings of the National Academy of Sciences 111, 3955-3960 (2014).
22 Li, B. et al. An orthogonal array optimization of lipid-like nanoparticles for mRNA delivery in vivo. Nano letters 15, 8099-8107 (2015).
23 Tsou, Y. H. et al. Dopant-Free Hydrogels with Intrinsic Photoluminescence and Biodegradable Properties. Advanced Functional Materials 28, 1802607 (2018).
24 Ortiz, C. et al. Novozym 435: the “perfect” lipase immobilized biocatalyst? Catalysis Science & Technology 9, 2380-2420 (2019).
25 Basso, A. & Serban, S. Industrial applications of immobilized enzymes—A review. Molecular Catalysis 479, 110607 (2019).
26 Kundys, A., Bialecka-Florjariczyk, E., Fabiszewska, A. & Malajowicz, J. Candida antarctica lipase B as catalyst for cyclic esters synthesis, their polymerization and degradation of aliphatic polyesters. Journal of Polymers and the Environment 26, 396-407 (2018).
27 Cheng, Q. et al. Dendrimer-based lipid nanoparticles deliver therapeutic FAH mRNA to normalize liver function and extend survival in a mouse model of hepatorenal tyrosinemia type I. Advanced materials 30, 1805308 (2018).
28 Miao, L. et al. Delivery of mRNA vaccines with heterocyclic lipids increases antitumor efficacy by STING-mediated immune cell activation. Nature biotechnology 37, 1174-1185 (2019).
29 Kauffman, K. J. et al. Optimization of lipid nanoparticle formulations for mRNA delivery in vivo with fractional factorial and definitive screening designs. Nano letters 7300-7306 (2015).
30 Hassett, K. J. et al. Impact of lipid nanoparticle size on mRNA vaccine immunogenicity. Journal of Controlled Release 335, 237-246 (2021).
31 Chen, D. et al. Rapid discovery of potent siRNA-containing lipid nanoparticles enabled by controlled microfluidic formulation. Journal of the American Chemical Society 134, 6948-6951 (2012).
32 Whitehead, K. A. et al. Degradable lipid nanoparticles with predictable in vivo siRNA delivery activity. Nature communications 5, 1-10 (2014).
33 Swingle, K. L. et al. Amniotic fluid stabilized lipid nanoparticles for in utero intra-amniotic mRNA delivery. Journal of Controlled Release (2021).
34 Zhao, P. et al. Long-term storage of lipid-like nanoparticles for mRNA delivery. Bioactive materials 5, 358-363 (2020).
35 Chen, C., Han, D., Cai, C. & Tang, X. An overview of liposome lyophilization and its future potential. Journal of controlled release 142, 299-311 (2010).
36 Heyes, J., Palmer, L., Bremner, K. & MacLachlan, I. Cationic lipid saturation influences intracellular delivery of encapsulated nucleic acids. Journal of controlled release 107, 276-287 (2005).
37 Chen, S. et al. Influence of particle size on the in vivo potency of lipid nanoparticle formulations of siRNA. Journal of Controlled Release 235, 236-244 (2016).

### Embodiments

Generally, embodiments herein relate to: new class of ionizable cationic lipid compounds for lipid nanoparticle structures or formulations suitable for gene delivery systems. The new class of ionizable cationic lipid compounds are referred to as amino alcohol mediated ionizable cationic lipid compounds, which are a reaction product of an amino alcohol and an even-numbered carbon-content (C8 to C18) lipid acid, which may be saturated or unsaturated.

In one or more embodiments, the amino alcohol is selected from the structures with assigned numbers 1 to 18 (of FIG. 5), as follows:

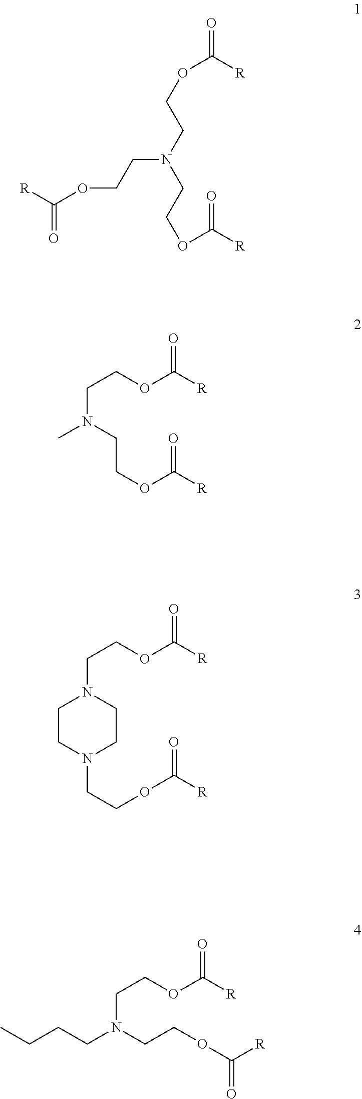

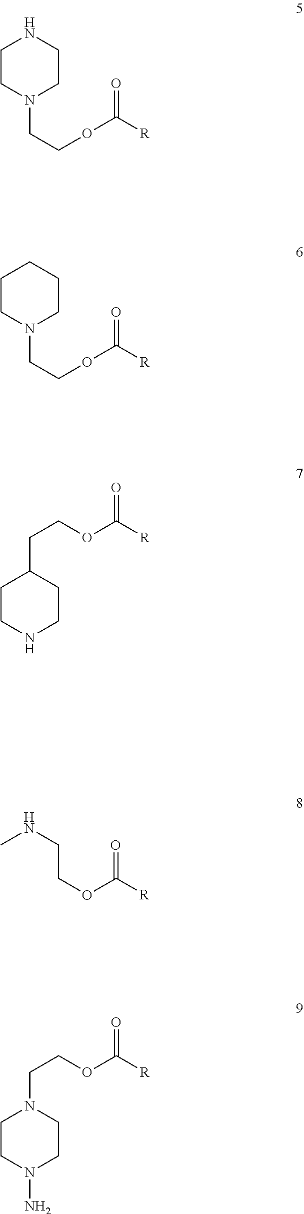

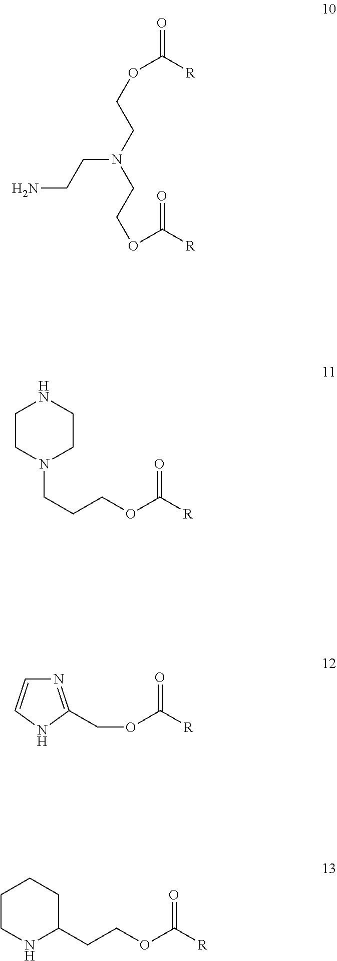

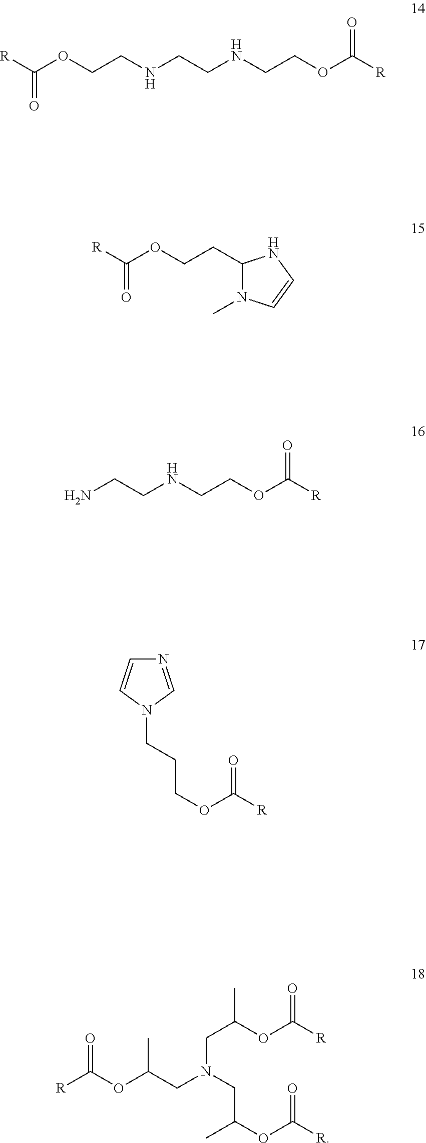

In one or more embodiments, the lipid acid is selected from the group consisting of octanoic acid (C8), decanoic acid (C10), dodecanoic acid (C12), tetradecanoic acid (C14), hexadecanoic acid (C16), octadecanoic acid (C18), oleic acid (C18:1), linoleic acid (C18:2), and/or combinations thereof.

In one or more embodiments, the amino alcohol mediated ionizable cationic lipid compound is selected from the following structures, which are the reaction product of amino alcohols with assigned numbers 1 to 18 of FIG. 5, and linoleic acid:

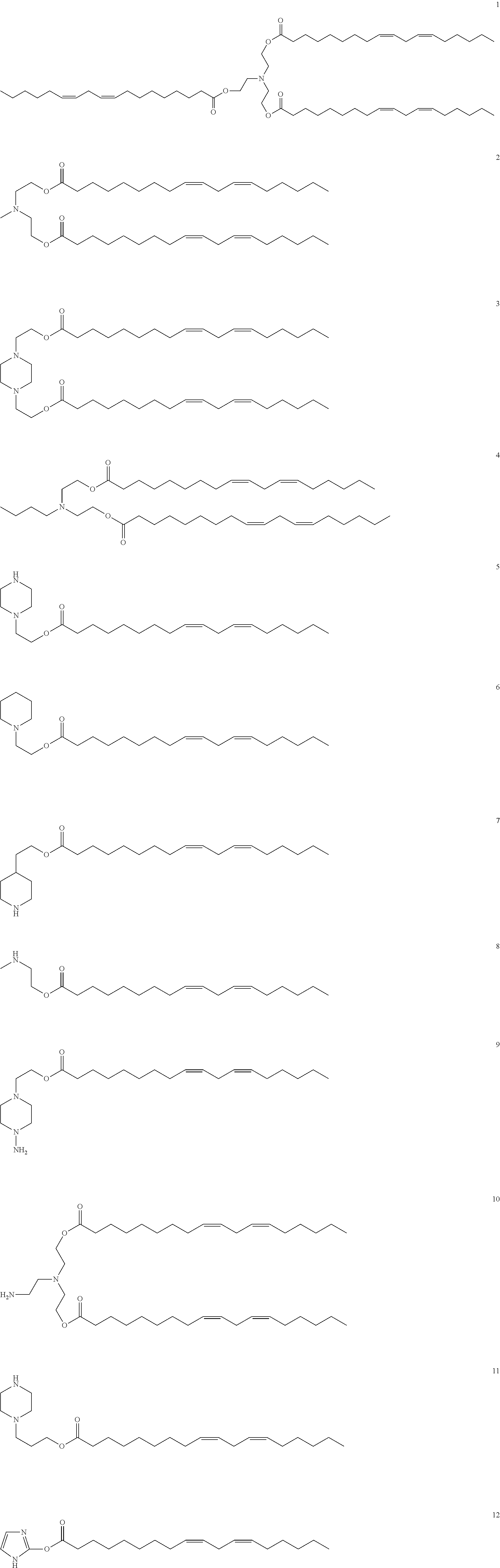

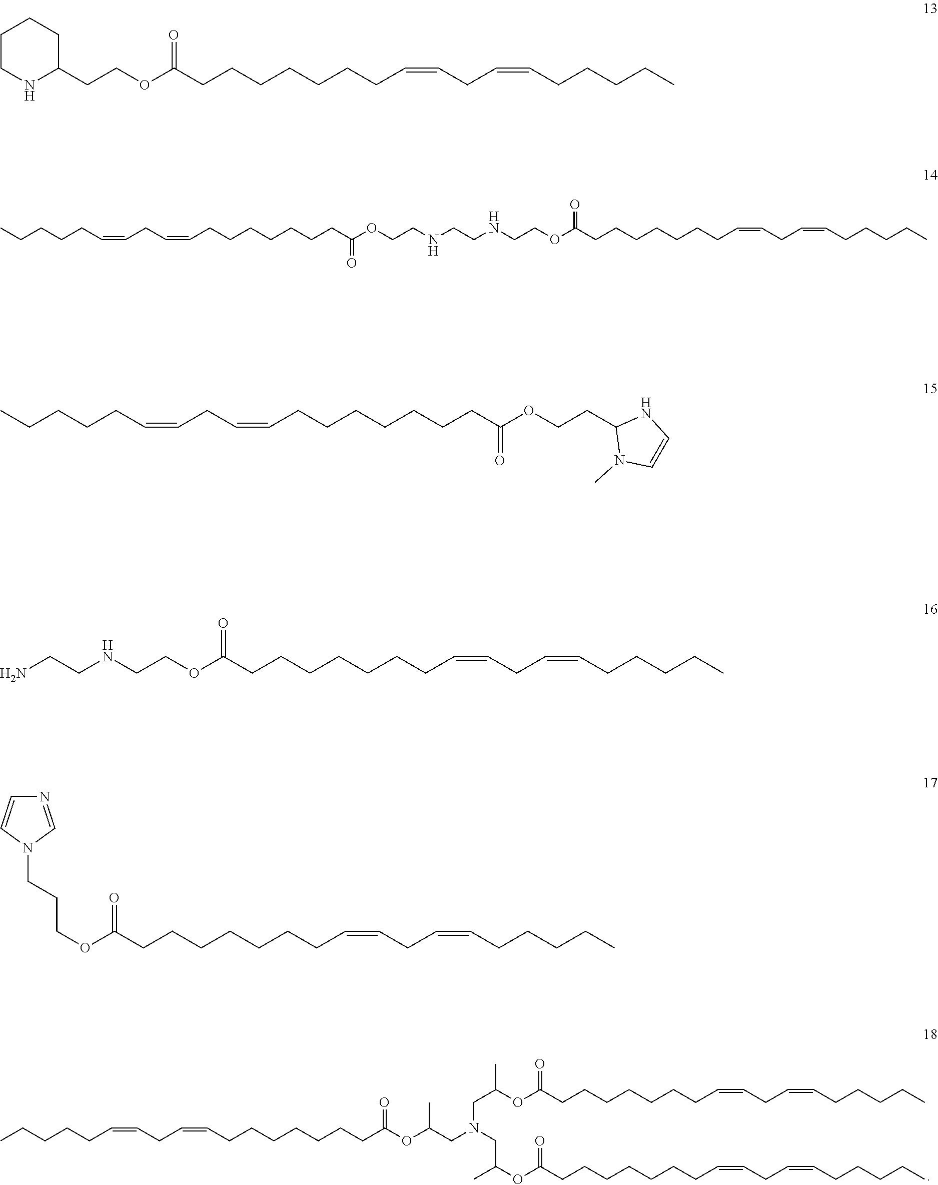

Lipid nanoparticle structures or formulations suitable for gene delivery systems herein comprise the amino alcohol mediated ionizable cationic lipid compounds and one or more lipid components. The amino alcohol mediated ionizable cationic lipid compounds and one or more lipid components assemble into lipid nanoparticle structures comprising an outer shell, which encapsulate nucleic acid agents or polynucleotides, which are desirable for gene therapy. The nucleic acid agents or polynucleotides have a size in a range of from 20 bp siRNAs to over 10,000 bp in the case of plasmids.

The one or more other lipid components generally include a neutral lipid, such as DOPE; a PEG-modified lipid (also referred to as PEG-lipid conjugate), such as DMG-PEG; and cholesterol.

In one or more embodiments, the lipid nanoparticles comprise: from 20 mol % to mol % of any amino alcohol mediated ionizable cationic lipid compound herein, from 20 mol % to 50 mol % of the neutral lipid, from 20 mol % to 50 mol % of the cholesterol, from mol % to 5 mol % of the PEG-modified lipid. In one or more embodiments, a total of the molar percentages of the amino alcohol mediated ionizable cationic lipid compound, the neutral lipid, the cholesterol, and the PEG-modified lipid is 100%.

In one or more embodiments, the therapeutic lipid nanoparticles: a mean particle size in a range of from 50 nm to 300 nm, and all values and subranges therebetween.

In one or more embodiments, the therapeutic lipid nanoparticles comprise: a pKa value in a range of 3.0 to 7.4, and all values and subranges therebetween.

In one or more embodiments, the therapeutic lipid nanoparticles comprise: a zeta potential in a range of from −20 mV to +20 mV, and all values and subranges therebetween.

Nanoparticle delivery systems herein comprise a plurality of any of the therapeutic lipid nanoparticles disclosed herein.

Further aspects herein provide methods of treating a genetic disorder, the method comprising administering a pharmaceutical composition to a subject in need thereof, the pharmaceutical composition comprising the lipid nanoparticle delivery system according to any embodiment herein. In one or more embodiments, the pharmaceutical composition is administered subcutaneously, intramuscularly, intravenously, or intraperitoneally.

Another aspect is a method of synthesis of an ionizable cationic lipid compound comprising: conducting one-step enzymatic esterification between hydroxyl groups (—OH) of an amino alcohol and carboxylic acid groups (—COOH) of a lipid acid in a reaction mixture including a Candida antarctica Lipase B-(CALB) catalyst to prepare the ionizable cationic lipid, which comprises an amino alcohol mediated ionizable cationic lipid compound; purifying the reaction mixture; and extracting the amino alcohol mediated ionizable cationic lipid compound. In one or more embodiments, the CALB catalyst is immobilized on acrylic resin. In an embodiment, the amino alcohol is dissolved in a solvent (e.g., THF) followed by addition of the lipid acid and the CALB catalyst, and the mixture is stirred. In one or more embodiments, the reactions are conducted at a temperature of 60° C.±30° C. under an inert atmosphere (e.g. nitrogen protection). For purification in one or more embodiments, the CALB catalyst is physically removed, e.g. by centrifuge, supernatant is collected and neutralized, e.g., by a saturated NaHCO₃ solution to neutralize excess lipid acid. Then, the amino alcohol mediated ionizable cationic lipid compound reaction product was extracted, e.g. by adding excess ethyl acetate following drying with anhydrous MgSO₄. Thereafter, in non-limiting embodiments, a clear lipid ethyl acetate solution is obtained after removing MgSO₄ by centrifuge and the lipid solution was concentrated by rotatory evaporating to remove the ethyl acetate and the resulting lipids were dried in vacuum.

Another aspect is a method of fabrication of a nanoparticle delivery system comprising: mixing an organic phase comprising an organic solvent and an amino alcohol mediated ionizable cationic lipid compound dissolved in the organic solvent with an aqueous phase comprising a nucleic acid agent dissolved in water in a receptacle to form a mixture using a microfluidic-chip device; and incubating the mixture to prepare lipid nanoparticles assembled from components of the organic phase. In one or more embodiments, a temperature of the mixture during the incubating is 25° C.±5° C. In one or more embodiments, the receptacle is one or more microchannels of a microfluidic chip device.

In one or more embodiments, a microfluidic chip device is utilized to fabricate the lipid nanoparticle (LNP) structures with substantially uniform particle size compared to mixing by pipetting. In one or more embodiments, the organic and aqueous phases are loaded in two different syringes for supply to the receptacle, namely microchannels of the microfluidic chip device. In an embodiment, a volume ratio of organic to aqueous phase is 1:3, which is achieved, for example, by setting respective syringe pump flow rates.

In some embodiments, the organic phase comprises ethanol as the organic solvent. In an embodiment, the organic phase comprises a mixture of the amino alcohol mediated ionizable cationic lipid in combination with one or more other lipid components. In an embodiment, the organic phase comprises a mixture of: the amino alcohol mediated ionizable cationic lipid, a neutral lipid, a PEG-modified lipid, and cholesterol. In an embodiment, the organic phase comprises a mixture of: a reaction product of: 1,4-Bis(2-hydroxyethyl) piperazine and linoleic acid (C18:2(9,12)), DOPE, cholesterol, and DMG-PEG 2000. In an embodiment, the organic phase comprises the reaction product of: 1,4-Bis(2-hydroxyethyl) piperazine and linoleic acid (C18:2(9,12)), DOPE, cholesterol, and DMG-PEG 2000 at a molar ratio of 40:40:25:0.5. In an embodiment, the aqueous phase is prepared in 25 mM NaOAc buffer (pH 5.0) with a gene of interest. In a detailed embodiment, incubating is conducted at room temperature before dialysis against 1× PBS in a Pur-A-Lyzer Midi Dialysis Kit (MWCO 3.5 kDa).

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

What is claimed is:

1. A lipid nanoparticle comprising:
an ionizable cationic lipid compound comprising a reaction product of: an amino alcohol and one or more lipid acids having from 4 to 26 carbons (C4-C26); and
one or more other lipid components selected from the group consisting of: a helper neutral lipid, a PEG-modified lipid, and/or cholesterol.

2. The lipid nanoparticle of claim 1, wherein the amino alcohol comprises two or more OH groups, and the one or more lipid acids comprise: octanoic acid (C8), decanoic acid (C10), dodecanoic acid (C12), tetradecanoic acid (C14), hexadecanoic acid (C16), octadecanoic acid (C18), oleic acid (C18:1), linoleic acid (C18:2), and/or combinations thereof.

3. The lipid nanoparticle of claim 1, wherein the amino alcohol comprises a piperzine derivative, and the one or more lipid acids comprises: octanoic acid (C8), decanoic acid (C10), dodecanoic acid (C12), tetradecanoic acid (C14), hexadecanoic acid (C16), octadecanoic acid (C18), oleic acid (C18:1), linoleic acid (C18:2), and/or combinations thereof.

4. The lipid nanoparticle of claim 1, wherein the amino alcohol comprises 1,4-Bis(2-hydroxyethyl) piperazine, the one or more lipid acids comprises linoleic acid (C18:2(9,12)); and the one or more other lipid components comprise: 1,2-dioleoyl-sn-glycero-3-phosphoethanolamine (DOPE), cholesterol, and 1,2-dimyristoyl-rac-glycero-3-methoxypolyethylene glycol (DMG-PEG).

5. The lipid nanoparticle of claim 1, wherein the helper neutral lipid comprises 1,2-dioleoyl-sn-glycero-3-phosphoethanolamine (DOPE); 1,2-di stearoyl-sn-glycero-3-phosphocholine (DSPC); and/or dioleoylphosphatidylcholine (DOPC).

6. The lipid nanoparticle of claim 1, wherein the PEG-modified lipid comprises 1,2-dimyristoyl-rac-glycero-3-methoxypolyethylene glycol (DMG-PEG).

7. The lipid nanoparticle of claim 1 comprising one of the following structures:

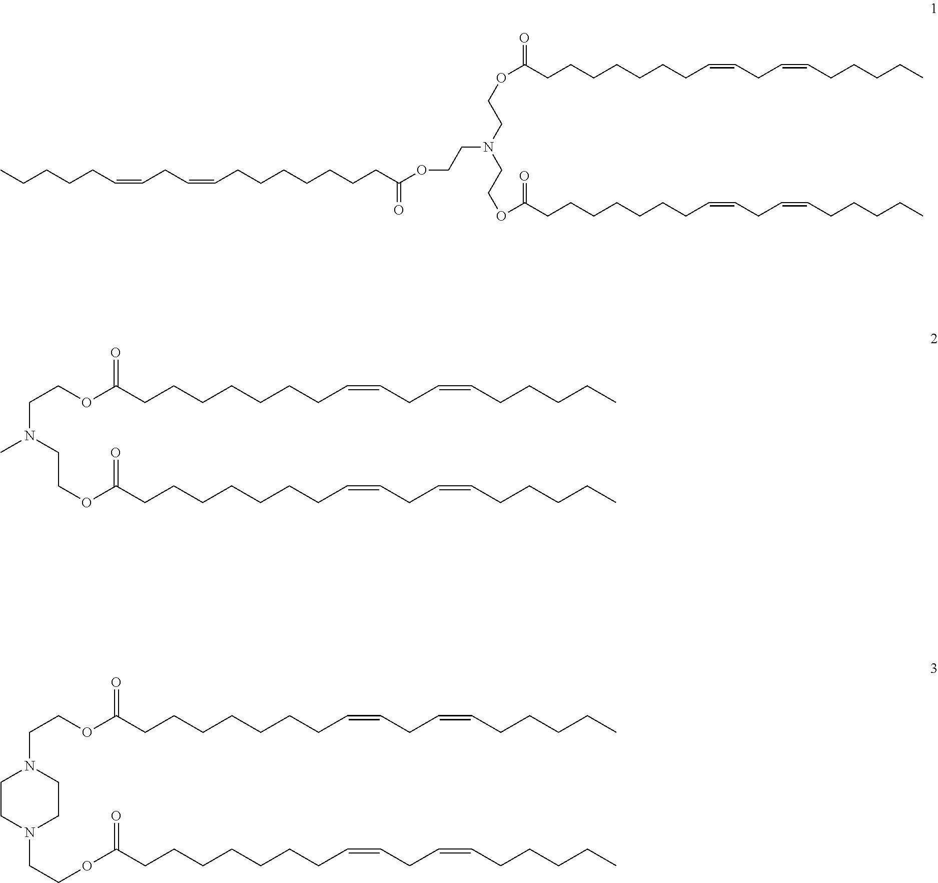

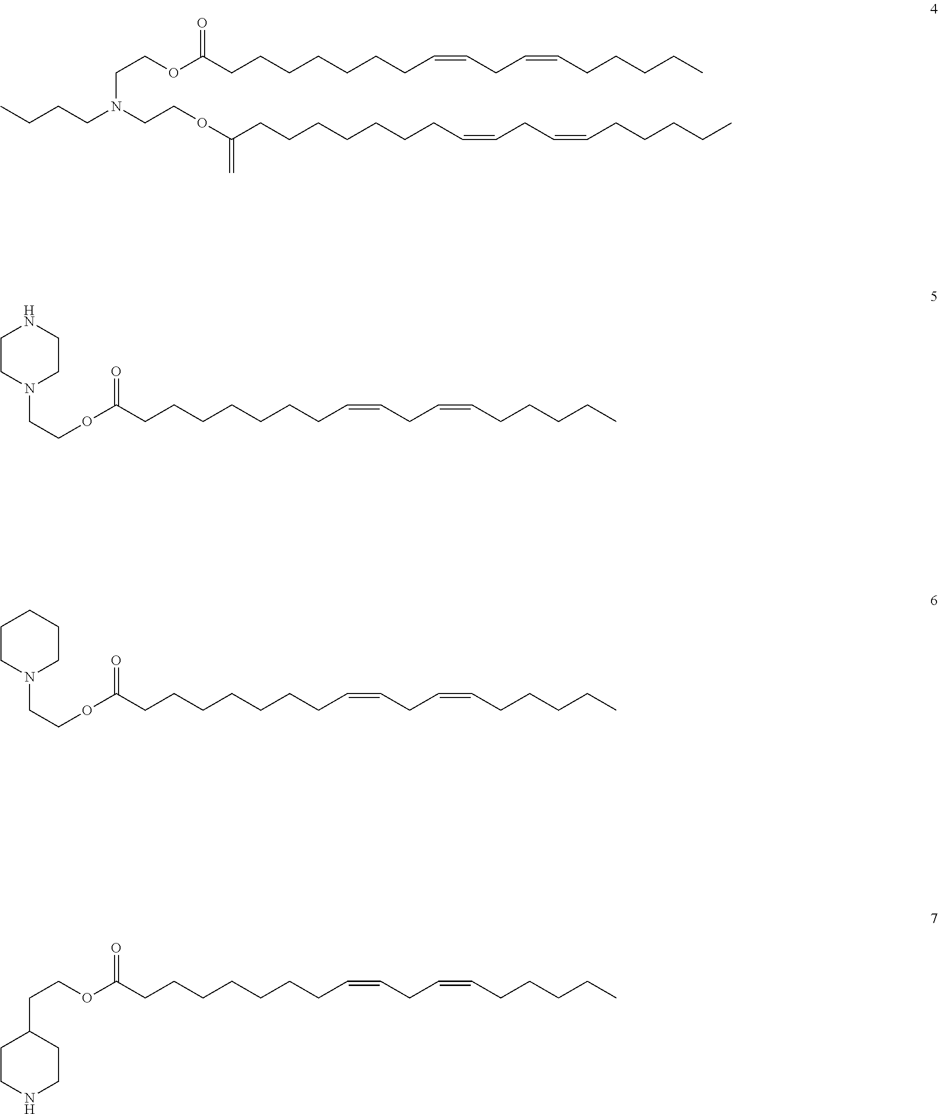 [SMILES: C=C(CCCCCCC/C=C\C/C=C\CCCCC)OCCN(CCCC)CCOC(=O)CCCCCCC/C=C\C/C=C\CCCCC ; CCCCC/C=C\C/C=C\CCCCCCCC(=O)OCCN1CCNCC1 ; CCCCC/C=C\C/C=C\CCCCCCCC(=O)OCCN1CCCCC1 ; CCCCC/C=C\C/C=C\CCCCCCCC(=O)OCCC1CCNCC1]

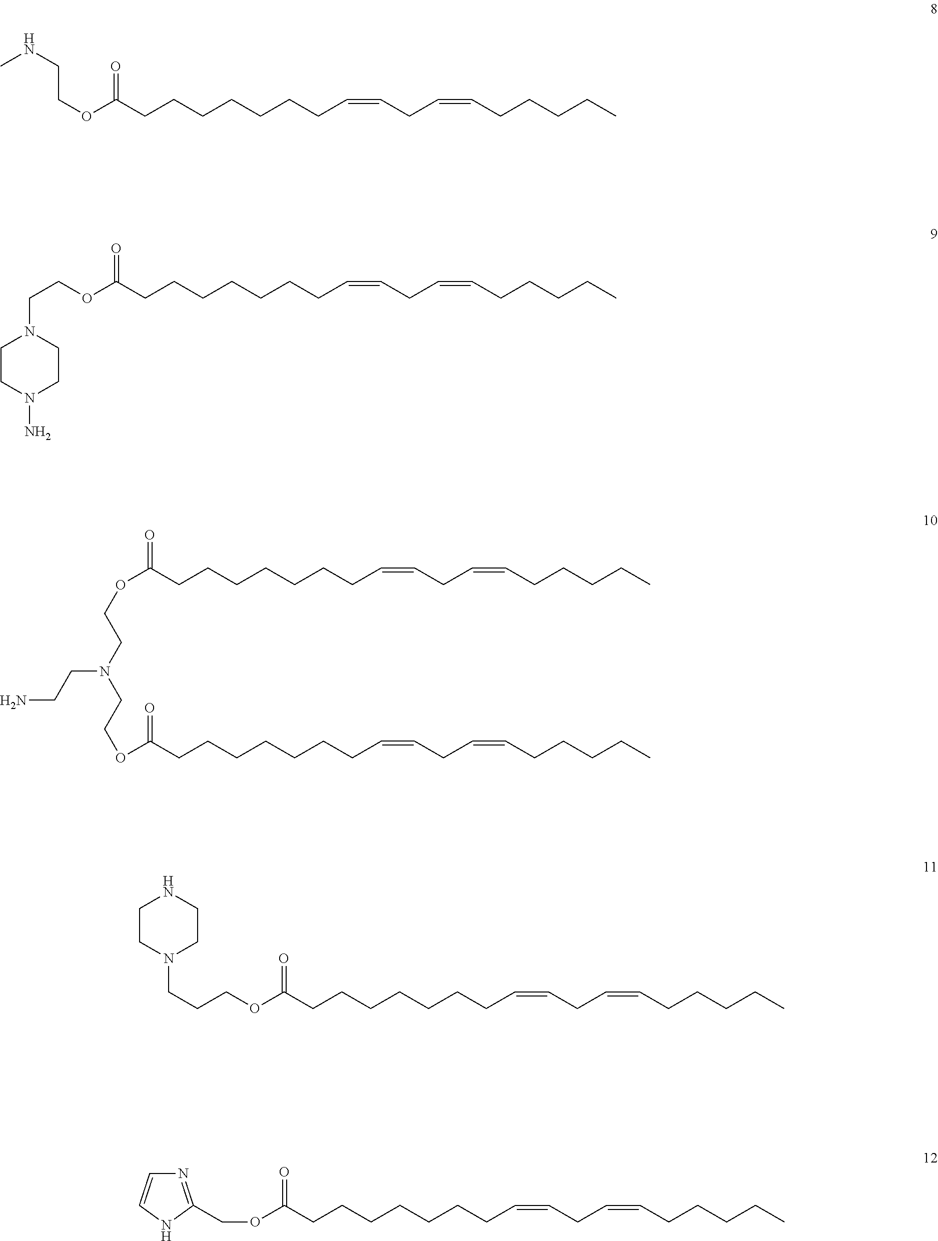 [SMILES: CCCCC/C=C\C/C=C\CCCCCCCC(=O)OCCNC ; CCCCC/C=C\C/C=C\CCCCCCCC(=O)OCCN1CCN(N)CC1 ; CCCCC/C=C\C/C=C\CCCCCCCC(=O)OCCN(CCN)CCOC(=O)CCCCCCC/C=C\C/C=C\CCCCC ; CCCCC/C=C\C/C=C\CCCCCCCC(=O)OCCCN1CCNCC1 ; CCCCC/C=C\C/C=C\CCCCCCCC(=O)OCc1ncc[nH]1]

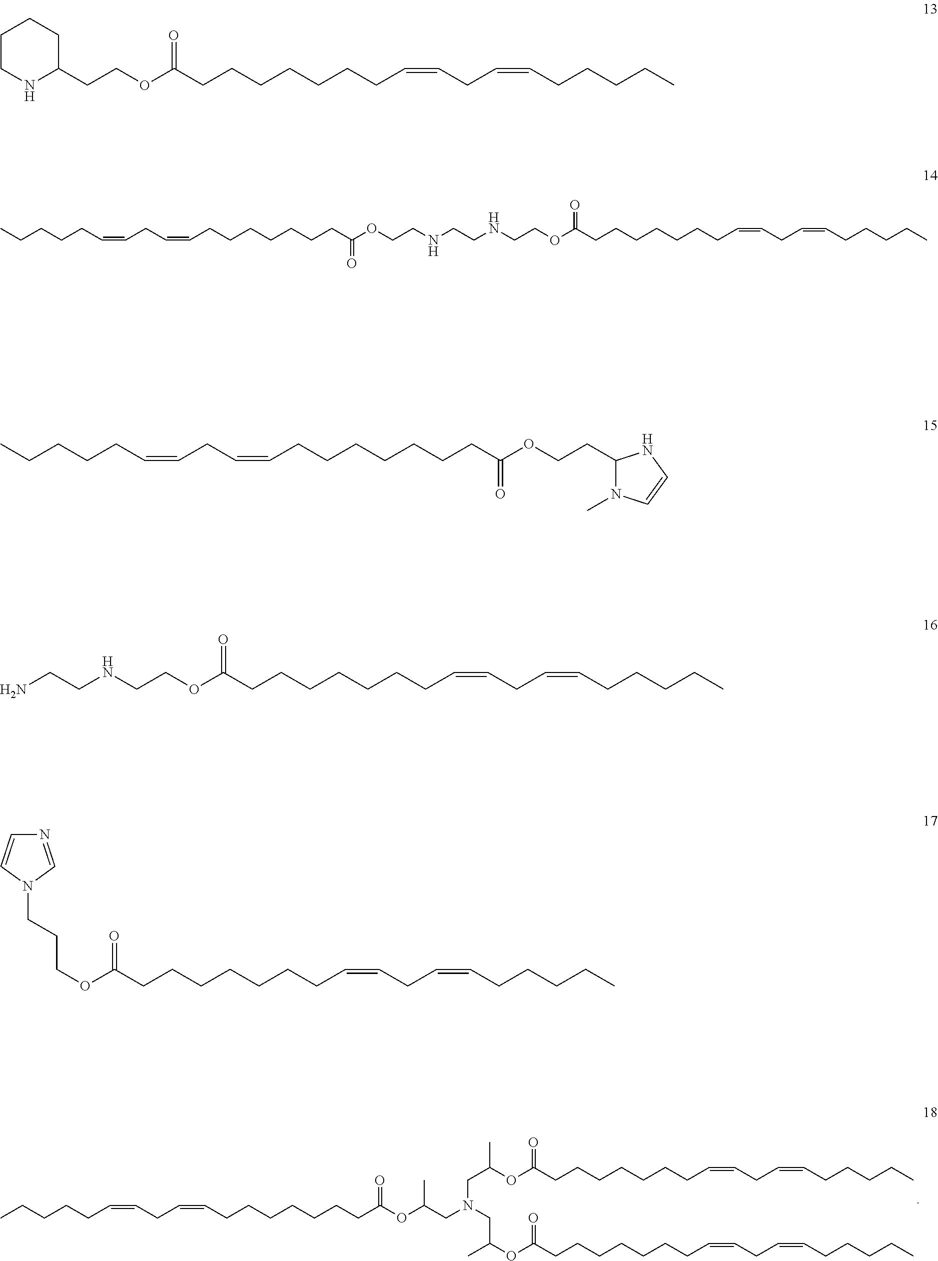

8. The lipid nanoparticle of claim 1, wherein the amino alcohol mediated ionizable cationic lipid compound comprises a reaction product of: 1,4-Bis(2-hydroxyethyl) piperazine and linoleic acid (C18:2(9,12)); and the one or more lipid components comprise: 1,2-dioleoyl-sn-glycero-3-phosphoethanolamine (DOPE), cholesterol, and 1,2-dimyristoyl-rac-glycero-3-methoxypolyethylene glycol (DMG-PEG).

9. The lipid nanoparticle of claim 8 comprising a molar ratio according to the following: 40:40:25:0.5 of the amino alcohol mediated ionizable cationic lipid compound: DOPE: cholesterol: DMG-PEG.

10. The lipid nanoparticle of claim 1 comprising a molecular weight in a range of from 200 to 2000 Daltons.

11. The lipid nanoparticle of claim 1, wherein the cationic lipid compound is synthesized via one-step Candida antarctica Lipase B-(CALB) esterification.

12. The lipid nanoparticle of claim 1 comprising: from 20 mol % to 60 mol % of the ionizable cationic lipid compound, from 20 mol % to 50 mol % of the helper neutral lipid, from 20 mol % to 50 mol % of cholesterol, and from 0.1 mol % to 5 mol % of the PEG-modified lipid.

13. The lipid nanoparticle of claim 12, wherein a total of the molar percentages of the ionizable cationic lipid compound, the helper neutral lipid, the cholesterol, and the PEG-modified lipid is 100 mol %.

14. A therapeutic lipid nanoparticle comprising: a lipid phase and a nucleic acid agent, the lipid phase comprising the lipid nanoparticle of claim 1.

15. A lipid nanoparticle delivery system comprising a plurality of the therapeutic lipid nanoparticles according to claim 14.

16. The nanoparticle delivery system of claim 15, wherein the nucleic acid agent comprises a DNA, an siRNA, a microRNA, an mRNA, a RNAi, a plasmid, or their antisense, single-stranded, double-stranded, or circular varieties.

17. The nanoparticle delivery system of claim 15, wherein the therapeutic lipid nanoparticle has a mean particle size in a range of from 50 nm to 300 nm.

18. The nanoparticle delivery system of claim 15, wherein the therapeutic lipid nanoparticle has a pKa value in a range of from 3.0 to 7.4.

19. The nanoparticle delivery system of claim 15, wherein the therapeutic lipid nanoparticle has a zeta potential in a range of from −40 mV to +40 mV.

20. The nanoparticle delivery system of claim 15 that is effective for sustained release of the nucleic acid agent.

21. The nanoparticle delivery system of claim 15, wherein a weight ratio of the lipid phase:nucleic acid agent is about 1:1 to about 1:100 (wt/wt).

22. A method of treating a genetic disorder, the method comprising administering a pharmaceutical composition to a subject in need thereof, the pharmaceutical composition comprising the lipid nanoparticle delivery system according to claim 15.

23. The method of claim 22, wherein the pharmaceutical composition is administered subcutaneously, intramuscularly, intravenously, or intraperitoneally.

24. A method of synthesis of an ionizable cationic lipid compound comprising:
conducting one-step enzymatic esterification between hydroxyl groups (—OH) of an amino alcohol and carboxylic acid groups (—COOH) of a lipid acid in a reaction mixture including a Candida antarctica Lipase B-(CALB) catalyst to prepare the ionizable cationic lipid, which comprises an amino alcohol mediated ionizable cationic lipid compound;
purifying the reaction mixture; and
extracting the amino alcohol mediated ionizable cationic lipid compound.

25. The method of claim 24, wherein the amino alcohol mediated ionizable cationic lipid compound comprises a reaction product of: 1,4-Bis(2-hydroxyethyl) piperazine and linoleic acid (C18:2(9,12)).

26. The method of claim 24, wherein the amino alcohol comprises two or more OH groups, and the one or more lipid acids comprises: octanoic acid (C8), decanoic acid (C10), dodecanoic acid (C12), tetradecanoic acid (C14), hexadecanoic acid (C16), octadecanoic acid (C18), oleic acid (C18:1), linoleic acid (C18:2), and/or combinations thereof.

27. The method of claim 24, wherein the amino alcohol comprises a piperzine derivative, and the one or more lipid acids comprises: octanoic acid (C8), decanoic acid (C10), dodecanoic acid (C12), tetradecanoic acid (C14), hexadecanoic acid (C16), octadecanoic acid (C18), oleic acid (C18:1), linoleic acid (C18:2), and/or combinations thereof.

28. A method of fabrication of a nanoparticle delivery system comprising:
mixing an organic phase comprising an organic solvent and an amino alcohol mediated ionizable cationic lipid compound dissolved in the organic solvent with an aqueous phase comprising a nucleic acid agent dissolved in water in a receptacle to form a mixture using a microfluidic-chip device; and
incubating the mixture to prepare therapeutic lipid nanoparticles assembled from components of the organic phase and the nucleic acid agents.

29. The method of claim 28, wherein a temperature of the mixture during the incubating is 20°±5° C.

30. The method of claim 28, wherein the receptacle is one or more microchannels of the microfluidic chip device.
